(19) United States
(12) Patent Application Publication
(10) Pub. No.: US 2006/0058250 A1
(43) Pub. Date: Mar. 16, 2006

(54) Methods of treating proliferative skin diseases using carbazole derivatives

(75) Inventors: Samuel R. Denmeade, Ellicott City, MD (US); Robert L. Hudkins, Chester Springs, PA (US)

Correspondence Address: CEPHALON, INC., 41 MOORES ROAD, PO BOX 4011, FRAZER, PA, 19355, US

(73) Assignee: Cephalon, Inc., Frazer, PA (US)

(21) Appl. No.: 11/222,409

(22) Filed: Sep. 8, 2005

Related U.S. Application Data
(60) Provisional application No. 60/609,203, filed on Sep. 10, 2004.

(51) Int. Cl. A61K 31/7056; A61K 31/407

(52) U.S. Cl. 514/43; 514/410

(57) ABSTRACT

The present invention relates to a method of treating a proliferative skin disease, comprising administering a therapeutically effective amount of a trk inhibitor.

### FIELD OF THE INVENTION

The present invention relates to the field of treating proliferative skin diseases. In particular, the invention relates to the use of trk inhibitors, including fused pyrrolocarbazole derivatives in the treatment of proliferative skin diseases, including psoriasis.

### BACKGROUND OF THE INVENTION

Abnormalities in the rate of cell proliferation in keratinocytes, sometimes combined with abnormal rates of apoptosis and/or inflammation, can result in hyperproliferation that manifests in a number of proliferative skin disorders including actinic keratosis, basal cell carcinoma, squamous cell carcinoma, fibrous histiocytoma, dermatofibrosarcoma protuberans, hemangioma, nevus flammeus, xanthoma, Kaposi's sarcoma, mastocytosis, mycosis fungoides, lentigo, nevocellular nevus, lentigo maligna, malignant melanoma, metastatic carcinoma and various forms of psoriasis, including psoriasis vulgaris and psoriasis eosinophilia.

One particular type of hyperproliferative skin disease is psoriasis, which is a chronic, genetically influenced, skin disorder that affects 1 to 3 percent of the world's population. Psoriasis is a disabling disease with a social and economic impact that is underestimated by physicians and other health care providers. There are several types of psoriasis, including pustular, guttate, arthritic variants, and chronic plaque psoriasis, its most common form. Onset of psoriasis is associated with visible manifestations, which are circumscribed, thickened, scaly plaques that may be pruritic and are found most often on the elbows, knees, buttocks, scalp, and sites of local trauma. The severity of involvement can be estimated by the Psoriasis Area and Severity Index, which takes into account the size of the area involved, redness, thickness, and scaling.

One form of psoriasis, plaque psoriasis, is characterized pathologically by hyperproliferation of the epidermis and inflammation of the epidermis and dermis. The proliferative activity of psoriatic epidermis is much greater than normal; the migration of keratinocytes from the basal layer to the epidermal surface is more rapid, and the duration of the cell cycle of keratinocytes is shortened.

There is at present no cure for psoriasis, only suppressive therapy. The treatments available for psoriasis currently include topical, phototherapy and systemic therapy. Patients typically undergo treatment with topical agents for mild to moderate forms of psoriasis, and a proportion of patients progress through phototherapy to systemic treatments as the disease grows more severe.

Examples of topical treatments include anthralin, coal tar, corticosteroid ointment, vitamin based-creams such as tazarotene and calcipotriene, pimecrolimus (Elidel®) and tacrolimus (Prograf®). Despite the benefits associated with localized topical treatments, the topical treatments have severe limitations including: coal tar—unpleasant odor, causes irritation, can form acneiform eruption on normal skin, and linked with skin cancer; anthralin—can stain the skin and clothes, and irritates skin; corticosteroids—thinning of the skin, striae, masking of local infections, hypopigmentation, and tolerance (tachyphylaxis) to the anti-inflammatory action of the treatment; and calcipotriene—rate of relapse and the safety associated with long-term treatment not known.

Some patients elect to undergo phototherapy, such as with an excimer laser (high intensity UVB) or more conventional UVB and UVA. However, these treatments can cause pain and irritation and may increase the long-term risk of skin cancer. Furthermore, phototherapy is clinic-intensive as treatment is typically performed at a clinic or doctor's office by a technician. This requires a lot of effort for patients to undergo and therefore, this type of treatment is undesirable.

Although the majority of patients with psoriasis are treated with topical agents and phototherapy, some may require more aggressive treatment. More aggressive therapy may be indicated when treating large areas (more than 20 percent of the body surface) topically is impractical because of the inconvenience and expense, or when the patient has psoriasis unresponsive to topical therapy, is occupationally disabled, or is affected psychologically by the disease. Systemic treatments include the retinoid acitretin (Soriatane®), cyclosporine (Neoral®), and methotrexate. These regimens may cause some toxic effects, and the therapeutic index of each must be evaluated repeatedly to avoid excessive risk in relation to the benefits. While methotrexate is an alternative treatment sought for severe conditions, adequate renal function is necessary because 85 percent of the drug is excreted through the kidneys, and patients with poor renal function have sustained increases in plasma drug concentrations, leading to acute side effects, including leucopenia and acute gastrointestinal or cutaneous erosions. The chief long-term side effect of methotrexate therapy is cirrhosis; patients with a history of liver disease or excessive alcohol intake and those with abnormal liver function should not receive the drug. Patients with extensive psoriasis who are treated with cyclosporine may see improvement; however, like other treatments for psoriasis, cyclosporine is not curative. The disease has been found to typically relapse within days or weeks after the discontinuation of treatment. Also, the side effects of cyclosporine include hypertension and impairment of renal function, which may be irreversible. The immunosuppressive properties of cyclosporine raise the possibility of an increased risk of cancer. Currently, available information indicates that cyclosporine should be given for no more than one year.

Normal keratinocytes express nerve growth factor (NGF) in a growth regulated fashion. NGF is known to bind to a low affinity (p75) and a high affinity receptor (trkA). Although not extensively researched, some recent studies have shown that NGF may play a role in psoriasis. One group reports that NGF levels in psoriatic keratinocytes are higher than levels in normal keratinocytes. NGF has also been reported to have a protective affect on keratinocytes. One study showed that K-252a, an inhibitor of tyrosine phosphorylation, can block an autocrine NGF loop and result in keratinocyte apoptosis. The same group found that K-252a treatment resulted in a noticeable improvement in the skin condition in a SCID-mouse-human skin model of psoriasis. Raychaudhuri S P, et al. J. Invest. Dermatol. 122:812-819 (2004). Although such research shows interesting results, more investigation is required to elucidate the role of NGF in inflammation and hyperproliferation of keratinocytes.

There still remains a need for a new method of treating or alleviating proliferative skin disorders by administering a therapeutically effective composition. More specifically, there is a need for a method of treating or alleviating psoriasis. The present invention is directed to these remaining needs as well as other needs.

### SUMMARY OF THE INVENTION

The present invention is directed to methods for treating proliferative skin diseases comprising administering a therapeutic composition of a compound that is a trk inhibitor.

In one embodiment of the present invention the trk inhibitor has the formula (A1): 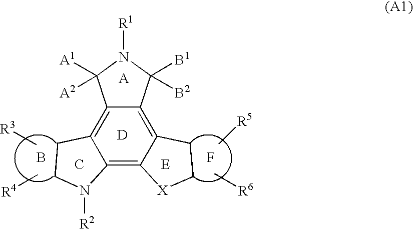
wherein the constituent members are defined infra.

In another aspect, the present invention is directed to pharmaceutical compositions which comprises one or more pharmaceutically acceptable excipients and a therapeutically effective amount of a trk inhibitor, including the fused pyrrolocarbazole compounds of the present invention, more fully described below.

### DETAILED DESCRIPTION OF THE INVENTION

The present invention is directed to methods for treating proliferative skin diseases comprising administering a therapeutic composition including an active agent having the Formula (A1): 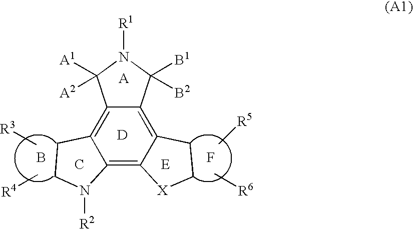
or a stereoisomer or pharmaceutically acceptable salt form thereof, wherein:
rings B and F, independently, are phenyl or heteroaryl;
R¹ is H; alkyl; aryl; arylalkyl; heteroaryl; heteroarylalkyl; —COR⁹; —OR¹⁰; —CONR⁷R⁸; —NR⁷R⁸; —(CH₂)ₚNR⁷R⁸; —(CH₂)ₚOR¹⁰; —O(CH₂)ₚOR¹⁰; or —O(CH₂)ₚNR⁷R⁸;
R² is H; —SO₂R⁹; —CO₂R⁹; —COR⁹; alkyl having 1 to 8 carbons; alkenyl having 2 to 8 carbons; alkynyl having 2 to 8 carbons; or a monosaccharide having 5 to 7 carbons;
   wherein each hydroxyl group of the monosaccharide, independently, is optionally replaced by an alkyl having 1 to 4 carbons, alkylcarbonyloxy having 2 to 5 carbons or alkoxy having 1 to 4 carbons; and
   wherein the alkyl, alkenyl, or alkynyl groups are optionally substituted with one to three R²⁷ groups;
R³, R⁴, R⁵ and R⁶, independently, are H; aryl; heteroaryl; F; Cl; Br; I; —CN; CF₃; —NO₂; —OR¹⁰; —O(CH₂)ₚNR⁷R⁸; —OCOR⁹; —OCONHR⁹; —CH₂OR¹⁴; —NR⁷R⁸; —NR¹⁰COR⁹; —NR¹⁰CONR⁷R⁸; —S(O)_{y}R¹¹; —CO₂R⁹; —COR⁹; —CONR⁷R⁸; —CHO; —CH═NOR¹¹; —CH═NR⁹; —CH═NNR¹²R¹³; —(CH₂)ₚS(O)_{y}R⁹; —CH₂SR¹⁵; —CH₂S(O)_{y}R¹⁴; —(CH₂)ₚNR⁷R⁸; —(CH₂)ₚNHR¹⁴; alkyl having 1 to 8 carbons; alkenyl having 2 to 8 carbons; or alkynyl having 2 to 8 carbons;
   wherein the alkyl, alkenyl, or alkynyl groups are optionally substituted with one to three R²⁷ groups;
X is:
   alkylene having 1 to 3 carbons optionally substituted with at least one of OH; ═O; ═NOR¹¹; OR¹¹; —OCOR⁹; —OCONR⁷R⁸; —O(CH₂)ₚNR⁷R⁸; —O(CH₂)ₚOR¹⁰; aryl; arylalkyl; heteroaryl; —SO₂R; —CO₂R⁹; —COR⁹; alkyl having 1 to 8 carbons; alkenyl having 2 to 8 carbons; alkynyl having 2 to 8 carbons; or a monosaccharide having 5 to 7 carbons;
      wherein each hydroxyl group of the monosaccharide, independently, is optionally replaced by an alkyl having 1 to 4 carbons, alkylcarbonyloxy having 2 to 5 carbons or alkoxy having 1 to 4 carbons; and
      wherein the alkyl, alkenyl, or alkynyl groups are optionally substituted with one to three R²⁷ groups;
   —O—; —S(O)_{y}—; N(R¹⁶); —CH₂Z—; —Z—CH₂—; or —CH₂ZCH₂—;
      wherein Z is C(OR¹¹)(R¹¹), O, S, C(═O), C(═NOR¹¹), or NR¹¹; or CHR¹⁶;
   wherein R¹⁶ and R² can optionally be combined together to form a linking furan via its 2 and 5 positions and wherein positions 2 and 5 of the linking furan are optionally substituted with R²⁸ and R²⁹, respectively; and position 3 of the linking furan is disubstituted with R¹⁷ and R¹⁸;
A¹ and A², independently, are H, —OR¹¹, —SR¹¹, or —N(R¹¹)₂; or, combined together, form a moiety that is ═O, ═S, or ═NR¹¹; and
B¹ and B² independently, are H, —OR¹¹, —SR¹¹, or —N(R¹¹)₂; or, combined together, form a moiety that is ═O, ═S, or ═NR¹¹;
   with the proviso that at least one of the pair of A¹ and A², or B¹ and B² is combined together to form ═O;
R¹ and R⁸, independently, are H or alkyl of 1 to 4 carbons, or, together with the nitrogen to which they are attached, form a 5 to 7 membered heterocycloalkyl;
R⁹ is alkyl having 1 to 4 carbons, aryl, or heteroaryl;
R¹⁰ is H or alkyl having 1 to 4 carbons;
R¹¹ is H, alkyl having 1 to 4 carbons, aryl having 6 to 10 carbons, or heteroaryl;
R¹² and R¹³, independently, are H, alkyl, aryl having 6 to 10 carbons, or heteroaryl; or, together with the nitrogen to which they are attached, form a 5 to 7 membered heterocycloalkyl;
R¹⁴ is the residue of an amino acid after the hydroxyl group of the carboxyl group is removed;
R¹⁵ is alkyl having 1 to 4 carbons;
R¹⁶ is lower alkyl, aryl, or heteroaryl;
R¹⁷ is OH, O-n-alkyl having 1 to 6 carbons, or O-acyl having 2 to 6 carbons;
R¹⁸ is H, alkyl having 1 to 4 carbons, CONHC₆H₅, or CH₂Y
   wherein Y is OR¹⁹; SOR²⁰; NR²¹R²²; or SR²³; N₃; CO₂R¹⁵; S-Glc; CONR²⁴R²⁵; CH═NNHCONH₂; CONHOR¹⁰; CH═NOR¹⁰; CH═NNHC(═NH)NH₂; 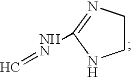
      CH═NN(R²⁶)₂; or CH₂NHCONHR¹⁶; or
R¹⁷ and R¹⁸ can optionally be combined together to form —CH₂NHCO₂—, —CH₂OC(CH₃)₂O—, ═O, or —CH₂N(CH₃)CO₂—; and
R¹⁹ is H, alkyl having 1 to 4 carbons, or acyl having 2 to 5 carbons;
R²⁰ is alkyl having 1 to 4 carbons, aryl, or a heterocycloalkyl group including a nitrogen atom;
R²¹ and R²², independently, are H, alkyl having 1 to 4 carbons, Pro, Ser, Gly, Lys, or acyl having 2 to 5 carbons, with the proviso that only one of R²¹ and R²² is Pro, Ser, Gly, Lys or acyl;
R²³ is an aryl, alkyl having 1 to 4 carbons, or a heterocycloalkyl group that includes a nitrogen atom;
R²⁴ and R²⁵, independently, are H; alkyl having 1 to 6 carbons; phenyl; or hydroxyalkyl of 1-6 carbons; or, together with the nitrogen to which they are attached, form a 5 to 7 membered heterocycloalkyl;
R²⁶ is aryl;
R²⁷ is aryl; heteroaryl; F; Cl; Br; I; —CN; —NO₂; —OR¹⁰; —O(CH₂)ₚNR⁷R⁸; —OCOR⁹; —OCONHR⁹; O-tetrahydropyranyl; —NR⁷R⁸; —NR¹⁰COR⁹; —NR¹⁰CO₂R⁹; —NR¹⁰CONR⁷R⁸; —NHC(═NH)NH₂; —NR¹⁰SO₂R⁹; —S(O)_{y}R¹¹; —CO₂R⁹; —CONR⁷R⁸; —CHO; —COR⁹; —CH₂OR⁷; —CH═NNR¹²R¹³; —CH═NOR¹¹; —CH═NR⁹; —CH═NNHCH(N═NH)NH₂; —SO₂NR¹²R¹³; —PO(OR¹¹)₂; or —OR¹⁴;
R²⁸ and R²⁹, independently, is an alkyl having from 1 to 4 carbons, alkoxy having from 1 to 4 carbons, arylalkyl having from 6 to 10 carbons, —(CH₂)ₚOR¹⁰, —(CH₂)ₚOC(═O)NR⁷R⁸, or —(CH₂)ₚNR⁷R⁸;
p is an integer from 1 to 4; and
y is 0, 1 or 2.

Some embodiments of the present invention are represented by Formula (A2): 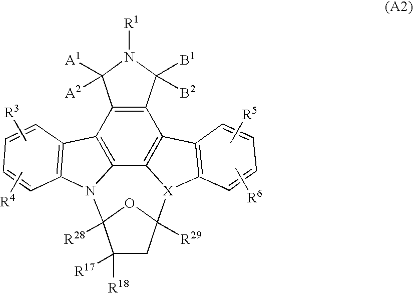
or a stereoisomer or pharmaceutically acceptable salt form thereof, wherein:
R¹ is H; alkyl; phenyl; arylalkyl having 7 to 10 carbons; 5-6 membered heteroaryl; heteroarylalkyl; —COR⁹; —OR¹⁰; —CONR⁷R⁸; —NR⁷R⁸; —(CH₂)ₚNR⁷R⁸; —(CH₂)ₚOR¹⁰; —O(CH₂)ₚOR¹⁰; or —O(CH₂)ₚNR⁷R⁸;
R³, R⁴, R⁵ and R⁶, independently, are H; phenyl; 5-6 membered heteroaryl; F; Cl; Br; I; —CN; CF₃; —NO₂; —OR¹⁰; —O(CH₂)ₚNR⁷R⁸; —OCOR⁹; —OCONHR⁹; —CH₂OR¹⁴; —NR⁷R⁸; —NR¹⁰COR⁹; —NR¹⁰CONR⁷R⁸; —S(O)_{y}R¹¹; —CO₂R⁹; —COR⁹; —CONR⁷R⁸; —CHO; —CH═NOR¹¹; —CH═NR; —CH═NNR¹²R¹³; —(CH₂)ₚS(O)_{y}R⁹; —CH₂SR¹⁵; —CH₂S(O)_{y}R¹⁴; —(CH₂)ₚNR⁷R⁸; —(CH₂)ₚNHR¹⁴; alkyl having 1 to 8 carbons; alkenyl having 2 to 8 carbons; or alkynyl having 2 to 8 carbons;
   wherein the alkyl, alkenyl, or alkynyl groups are optionally substituted with one to three R²⁷ groups;
X is —CH—, —O—, or N;
A¹ and A², independently, are H, —OR¹¹, —SR¹¹, or —N(R¹¹)₂; or, combined together, form a moiety that is ═O, ═S, or ═NR¹¹; and
B¹ and B² independently, are H, —OR¹¹, —SR¹¹, or —N(R¹¹)₂; or, combined together, form a moiety that is ═O, ═S, or ═NR¹¹;
   with the proviso that at least one of the pair of A¹ and A², or B¹ and B² is combined together to form ═O;
R⁷ and R⁸, independently, are H or alkyl of 1 to 4 carbons, or, together with the nitrogen to which they are attached, form a 5 to 7 membered heterocycloalkyl;
R⁹ is alkyl having 1 to 4 carbons, aryl, or heteroaryl;
R¹⁰ is H or alkyl having 1 to 4 carbons;
R¹¹ is H, alkyl having 1 to 4 carbons, aryl having 6 to 10 carbons, or heteroaryl;
R¹² and R¹³, independently, are H, alkyl, aryl having 6 to 10 carbons, or heteroaryl; or, together with the nitrogen to which they are attached, form a 5 to 7 membered heterocycloalkyl;
R¹⁴ is the residue of an amino acid after the hydroxyl group of the carboxyl group is removed;
R¹⁵ is alkyl having 1 to 4 carbons;
R¹⁷ is OH, O-n-alkyl having 1 to 6 carbons, or O-acyl having 2 to 6 carbons;
R¹⁸ is H, alkyl having 1 to 4 carbons, CONHC₆H₅, or CH₂Y
   wherein Y is OR¹⁹; SOR²⁰; NR²¹R²²; or SR²³; N₃; CO₂R¹⁵; S-Glc; CONR²⁴R²⁵; CH═NNHCONH₂; CONHOR¹⁰; CH═NOR¹⁰; CH═NNHC(═NH)NH₂; 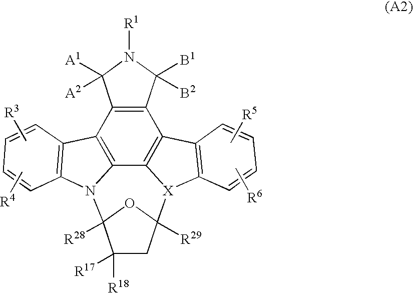
      CH═NN(R²⁶)₂; or CH₂NHCONHR¹⁶; or
R¹⁷ and R¹⁸ are combined together to form —CH₂NHCO₂—, —CH₂OC(CH₃)₂O—, ═O, or —CH₂N(CH₃)CO₂—; and
R¹⁹ is H, alkyl having 1 to 4 carbons, or acyl having 2 to 5 carbons;
R²⁰ is alkyl having 1 to 4 carbons, aryl, or a heterocycloalkyl group including a nitrogen atom;
R²¹ and R²², independently, are H, alkyl having 1 to 4 carbons, Pro, Ser, Gly, Lys, or acyl having 2 to 5 carbons, with the proviso that only one of R²¹ and R²² is Pro, Ser, Gly, Lys or acyl;
R²³ is an aryl, alkyl having 1 to 4 carbons, or a heterocycloalkyl group that includes a nitrogen atom;
R²⁴ and R²⁵, independently, are H; alkyl having 1 to 6 carbons; phenyl; or hydroxyalkyl of 1-6 carbons; or, together with the nitrogen to which they are attached, form a 5 to 7 membered heterocycloalkyl;
R²⁶ is aryl;
R²⁷ is aryl; heteroaryl; F; Cl; Br; I; —CN; —NO₂; —OR¹⁰; —O(CH₂)ₚNR⁷R⁸; —OCOR⁹; —OCONHR⁹; O-tetrahydropyranyl; —NR⁷R⁸; —NR¹⁰COR⁹; —NR¹⁰CO₂R⁹; —NR¹⁰CONR⁷R⁸; —NHC(═NH)NH₂; —NR¹⁰SO₂R⁹; —S(O)_{y}R¹¹; —CO₂R⁹; —CONR⁷R⁸; —CHO; —COR⁹; —CH₂OR⁷; —CH═NNR¹²R¹³; —CH═NOR¹¹; —CH═NR⁹; —CH═NNHCH(N═NH)NH₂; —SO₂NR¹²R¹³; —PO(OR¹¹)₂; or —OR¹⁴;
R²⁸ and R²⁹, independently, is an alkyl having from 1 to 4 carbons, alkoxy having from 1 to 4 carbons, arylalkyl having from 6 to 10 carbons, —(CH₂)ₚOR¹⁰, —(CH₂)ₚOC(═O)NR⁷R⁸, or —(CH₂)ₚNR⁷R⁸;

p is an integer from 1 to 4; and

y is 0, 1 or 2.

Some embodiments of the present invention are represented by Formula (A3): 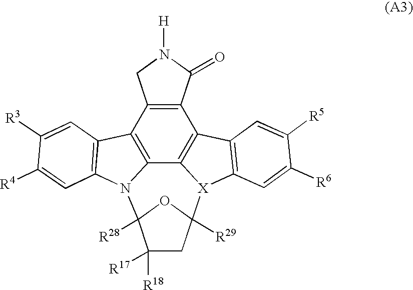
or a stereoisomer or pharmaceutically acceptable salt form thereof, wherein:
R¹ is H; alkyl; phenyl; arylalkyl having 7 to 10 carbons; 5-6 membered heteroaryl; heteroarylalkyl; —COR⁹; —OR¹⁰; —CONR⁷R⁸; —NR⁷R⁸; —(CH₂)ₚNR⁷R⁸; —(CH₂)ₚOR¹⁰; —O(CH₂)ₚOR¹⁰; or —O(CH₂)ₚNR⁷R⁸;
R³, R⁴, R⁵ and R⁶, independently, are H; phenyl; 5-6 membered heteroaryl; F; Cl; Br; I; —CN; CF₃; —NO₂; —OR¹⁰; —O(CH₂)ₚNR⁷R⁸; —OCOR⁹; —OCONHR⁹; —CH₂OR¹⁴; —NR⁷R⁸; —NR¹⁰COR⁹; —NR¹⁰CONR⁷R⁸; —S(O)_{y}R¹¹; —CO₂R⁹; —COR⁹; —CONR⁷R⁸; —CHO; —CH═NOR¹¹; —CH═NR⁹; —CH═NNR¹²R¹³; —(CH₂)ₚS(O)_{y}R⁹; —CH₂SR¹⁵; —CH₂S(O)_{y}R¹⁴; —(CH₂)ₚNR⁷R⁸; —(CH₂)ₚNHR¹⁴; alkyl having 1 to 8 carbons; alkenyl having 2 to 8 carbons; or alkynyl having 2 to 8 carbons;
   wherein the alkyl, alkenyl, or alkynyl groups are optionally substituted with one to three R²⁷ groups;
X is —CH—, —O—, or N;
A¹ and A², independently, are H, —OR¹¹, —SR¹¹, or —N(R¹¹)₂; or, combined together, form a moiety that is ═O, ═S, or ═NR¹¹; and
B¹ and B² independently, are H, —OR¹¹, —SR¹¹, or —N(R¹¹)₂; or, combined together, form a moiety that is ═O, ═S, or ═NR¹¹;
   with the proviso that at least one of the pair of A¹ and A², or B¹ and B² is combined together to form ═O;
R⁷ and R⁸, independently, are H or alkyl of 1 to 4 carbons, or, together with the nitrogen to which they are attached, form a 5 to 7 membered heterocycloalkyl;
R⁹ is alkyl having 1 to 4 carbons, aryl, or heteroaryl;
R¹⁰ is H or alkyl having 1 to 4 carbons;
R¹¹ is H, alkyl having 1 to 4 carbons, aryl having 6 to 10 carbons, or heteroaryl;
R¹² and R¹³, independently, are H, alkyl, aryl having 6 to 10 carbons, or heteroaryl; or, together with the nitrogen to which they are attached, form a 5 to 7 membered heterocycloalkyl;
R¹⁴ is the residue of an amino acid after the hydroxyl group of the carboxyl group is removed;
R¹⁵ is alkyl having 1 to 4 carbons;
R¹⁷ is OH, O-n-alkyl having 1 to 6 carbons, or O-acyl having 2 to 6 carbons;
R¹⁸ is H, alkyl having 1 to 4 carbons, CONHC₆H₅, or CH₂Y
   wherein Y is OR¹⁹; SOR²⁰; NR²¹R²²; or SR²³; N₃; CO₂R¹⁵; S-Glc; CONR²⁴R²⁵; CH═NNHCONH₂; CONHOR¹⁰; CH═NOR¹⁰; CH═NNHC(═NH)NH₂; 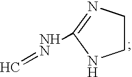
      CH═NN(R²⁶)₂; or CH₂NHCONHR¹⁶; or
R¹⁷ and R¹⁸ are combined together to form —CH₂NHCO₂—, —CH₂OC(CH₃)₂O—, ═O, or —CH₂N(CH₃)CO₂—; and
R¹⁹ is H, alkyl having 1 to 4 carbons, or acyl having 2 to 5 carbons;
R²⁰ is alkyl having 1 to 4 carbons, aryl, or a heterocycloalkyl group including a nitrogen atom;
R²¹ and R²², independently, are H, alkyl having 1 to 4 carbons, Pro, Ser, Gly, Lys, or acyl having 2 to 5 carbons, with the proviso that only one of R²¹ and R²² is Pro, Ser, Gly, Lys or acyl;
R²³ is an aryl, alkyl having 1 to 4 carbons, or a heterocycloalkyl group that includes a nitrogen atom;
R²⁴ and R²⁵, independently, are H; alkyl having 1 to 6 carbons; phenyl; or hydroxyalkyl of 1-6 carbons; or, together with the nitrogen to which they are attached, form a 5 to 7 membered heterocycloalkyl;
R²⁶ is aryl;
R²⁷ is aryl; heteroaryl; F; Cl; Br; I; —CN; —NO₂; —OR¹⁰; —O(CH₂)ₚNR⁷R⁸; —OCOR⁹; —OCONHR⁹; O-tetrahydropyranyl; —NR⁷R⁸; —NR¹⁰COR⁹; —NR¹⁰CO₂R⁹; —NR¹⁰CONR⁷R⁸; —NHC(═NH)NH₂; —NR¹⁰SO₂R⁹; —S(O)_{y}R¹¹; —CO₂R⁹; —CONR⁷R⁸; —CHO; —COR⁹; —CH₂OR⁷; —CH═NNR¹²R¹³; —CH═NOR¹¹; —CH═NR⁹; —CH═NNHCH(N═NH)NH₂; —SO₂NR¹²R¹³; —PO(OR¹¹)₂; or —OR¹⁴;
R²⁸ and R²⁹, independently, is an alkyl having from 1 to 4 carbons, alkoxy having from 1 to 4 carbons, arylalkyl having from 6 to 10 carbons, —(CH₂)ₚOR¹⁰, —(CH₂)ₚOC(═O)NR⁷R⁸, or —(CH₂)ₚNR⁷R⁸;
p is an integer from 1 to 4; and
y is 0, 1 or 2.

Some embodiments of the present invention are represented by Formula (A4): 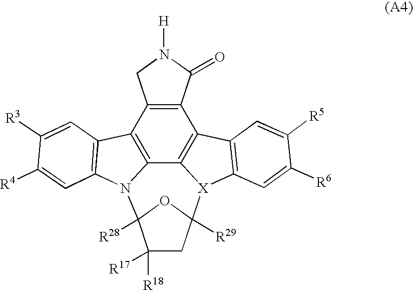
or a stereoisomer or pharmaceutically acceptable salt form thereof, wherein:
R³, R⁴, R⁵ and R⁶, independently, are H; phenyl; F; Cl; —OR¹⁰; —NR⁷R⁸; —CHO; —(CH₂)ₚNR⁷R⁸; or alkyl having 1 to 8 carbons;
   wherein the alkyl group is optionally substituted with one to three R²⁷ groups;
X is —CH— or N;
R⁷ and R⁸, independently, are H or alkyl of 1 to 4 carbons, or, together with the nitrogen to which they are attached, form a 5 to 7 membered heterocycloalkyl;
R⁹ is alkyl having 1 to 4 carbons, aryl, or heteroaryl;
R¹⁰ is H or alkyl having 1 to 4 carbons;
R¹¹ is H, alkyl having 1 to 4 carbons, aryl having 6 to 10 carbons, or heteroaryl;
R¹² and R¹³, independently, are H, alkyl, aryl having 6 to 10 carbons, or heteroaryl;
   or, together with the nitrogen to which they are attached, form a 5 to 7 membered heterocycloalkyl;
R¹⁴ is the residue of an amino acid after the hydroxyl group of the carboxyl group is removed;
R¹⁷ is OH, O-n-alkyl having 1 to 6 carbons, or O-acyl having 2 to 6 carbons;
R¹⁸ is H, alkyl having 1 to 4 carbons, CONHC₆H₅; CH₂OH; CH₂OCH; CH₂OCCH₃; CH₂NH₂; CO₂CH₃; CONR²⁴R²⁵;
R²⁴ and R²⁵, independently, are H; alkyl having 1 to 6 carbons; phenyl; or hydroxyalkyl of 1-6 carbons; or, together with the nitrogen to which they are attached, form a 5 to 7 membered heterocycloalkyl;
R²⁷ is aryl; heteroaryl; F; Cl; Br; I; —CN; —NO₂; —OR¹⁰; —O(CH₂)ₚNR⁷R⁸; —OCOR⁹; —OCONHR⁹; O-tetrahydropyranyl; —NR⁷R⁸; —NR¹⁰COR⁹; —NR¹⁰CO₂R⁹; —NR¹⁰CONR R⁸; —NHC(═NH)NH₂; —NR¹⁰SO₂R⁹; —S(O)_{y}R¹¹; —CO₂R⁹; —CONR⁷R⁸; —CHO; —COR⁹; —CH₂OR⁷; —CH═NNR¹²R¹³; —CH═NOR¹¹; —CH═NR⁹; —CH═NNHCH(N═NH)NH₂; —SO₂NR¹²R¹³; —PO(OR¹¹)₂; or —OR¹⁴;
R²⁸ and R²⁹, independently, is an alkyl having from 1 to 4 carbons, alkoxy having from 1 to 4 carbons, arylalkyl having from 6 to 10 carbons, —(CH₂)ₚOR¹⁰, —(CH₂)ₚOC(═O)NR⁷R⁸, or —(CH₂)ₚNR⁷R⁸;
p is an integer from 1 to 4; and
y is 0, 1 or 2.

Alternatively, some embodiments of the present invention are represented by Formula (A5): 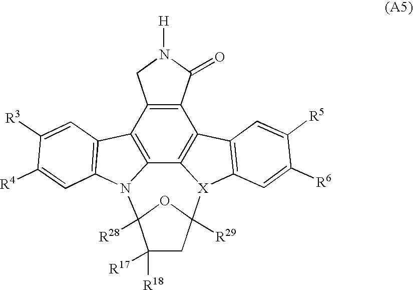
or a stereoisomer or pharmaceutically acceptable salt form thereof, wherein:
X is CH or N;
R³, R⁴, R⁵, and R⁶, independently, are H, Cl, alkyl of 1-4 carbons, —OR¹⁰, CH₂OH, CHO, NH₂, CH₂NH₂, CH₂OCH, CH₂OCCH₃, CONHC₆H₅, or CONH₂;
R¹⁰ is H or alkyl having 1 to 4 carbons;
R¹⁷ is OH, O-alkyl having 1 to 4 carbons;
R¹⁸ is H, CH₂OH, CO₂CH₃, CHOOCH₃, CHOOCH₂CH₃, CHOOCH₂CH₂CH₃, or CHOOCH(CH₃)₂; or
R²⁸ and R²⁹, independently, are H or CH₃.

One preferred embodiment of the present invention is represented by Formula (A6), which is also referred to as “lestaurtinib.” 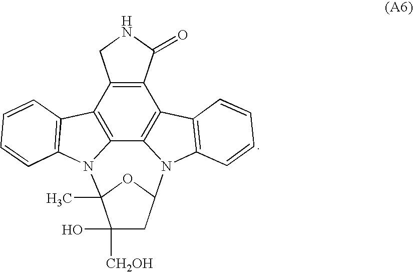
This compound, lestaurtinib, is indicated for the treatment of adult patients with moderate to severe chronic plaque psoriasis who are candidates for systemic therapy or phototherapy.

Another preferred embodiment of the present invention includes a compound of the formula (A7):

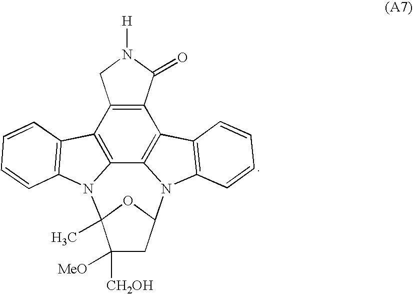

In one aspect of the present invention, the therapeutic composition includes an active agent having the formula listed in Table A, below.

TABLE A

FORMULA 1:

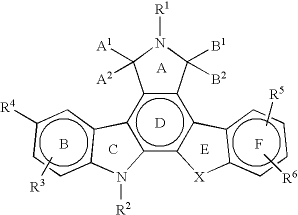

FORMULA 1a:

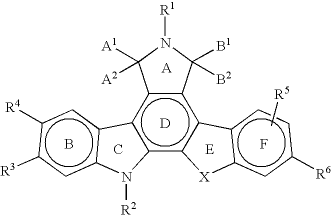

FORMULA 1b:

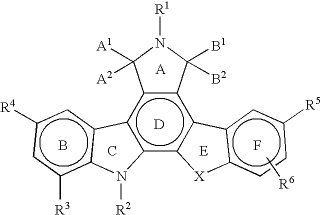

| Compound⁽¹⁾ | A¹A²⁽²⁾ | B¹B²⁽³⁾ | R² | R³ | R⁴ | X |
|---|---|---|---|---|---|---|
| 1-1 | O | O | H | H | H | CH₂ |
| 1-2 | H, H | O | H | H | H | CH₂ |
| 1-3 | O | H, H | H | H | H | CH₂ |
| 1-4 | O | O | CH₃ | H | H | CH₂ |
| 1-5⁽⁴⁾ | — | — | CH₃ | H | H | CH₂ |
| 1-6 | O | O | H | H | Br | CH₂ |
| 1-7 | O | O | H | H | F | CH₂ |
| 1-8⁽⁵⁾ | — | — | H | H | F | CH₂ |
| 1a-i | O | O | H | Cl | H | CH₂ |
| 1a-2 | — | — | H | Cl | H | CH₂ |
| 1-9 | H, H | O | H | H | Br | CH₂ |
| 1b-1 | O | O | H | CH₃ | H | CH₂ |
| 1-10 | O | O | H | H | Cl | CH₂ |
| 1-11 | O | H, H | H | H | Br | CH₂ |
| 1-12 | H, H | O | H | H | F | CH₂ |
| 1-13 | H, H | O | H | H | OCH₃ | CH₂ |
| 1a-3⁽⁷⁾ | O | O | H | H | H | CH₂ |
| 1b-2⁽⁸⁾ | O | O | H | H | H | CH₂ |
| 1-14 | O | O | H | H | H | CH₂CH₂ |
| 1-15 | O | O | H | H | H | CH═CH |
| 1-16 | O | H, H | H | H | H | CH₂CH₂ |
| 1-17 | H, H | O | H | H | H | CH₂CH₂ |
| 1-18 | O | H, H | H | H | H | CH₂CH₂ |
| 1-19 | H, H | O | H | H | H | CH₂CH₂ |
| 1-20 | O | O | H | H | H | S |
| 1-21 | O | O | H | H | H | O |
| 1-22 | O | H, H | H | H | F | CH₂CH₂ |
| 1-23 | O | H, H | H | H | F | CH₂ |
| 1-24 | H, H | O | H | H | HC═CHC₆H₅ | CH₂ |
| 1-25 | H, H | O | H | H | HC═CHCO₂C₂H₅ | CH₂ |
| 1-26 | H, H | O | CH₂CH═CH₂ | H | H | CH₂ |
| 1-27 | H, H | O | H | H | C₆H₅ | CH₂ |
| 1-28 | O | O | H | H | H | CO |
| 1-29 | H, H | O | CH₂CH₂OH | H | H | CH₂ |
| 1-30 | O | H,OH | H | H | H | CO |
| 1-31 | H, OH | O | H | H | H | CO |
| 1-32⁽¹²⁾ | H, H | O | H | H | HC═CH-2-pyr | CH₂ |
| 1a-4⁽⁹⁾ | O | O | H | H | H | CH₂CH₂ |
| 1-33 | H, H | O | H | H | HC═CH-4-pyr | CH₂ |
| 1-34 | H, H | O | H | H | H₂CCH₂-2-pyr | CH₂ |
| 1-35 | H, H | O | H | H | HC═CHCN | CH₂ |
| 1-36 | H, H | O | H | H | C≡CH | CH₂ |
| 1-37 | O | O | H | H | (CH₂)₄CH₃ | CH₂ |
| 1a-5⁽⁷⁾ | O | O | H | H | H | CH₂CH₂ |
| 1a-6⁽¹⁰⁾ | O | O | H | H | H | CH₂CH₂ |
| 1a-7⁽¹¹⁾ | O | O | H | H | H | CH₂CH₂ |
| 1-38 | H, OH | O | H | H | H | CH═CH |
| 1-39 | H, H | O | H | H | HC═CH-2-phthalimide | CH₂ |
| 1-40 | H, H | O | | | Iodo | CH₂ |
| 1-41 | O | H, H | H | H | HC═CH-2-pyr | CH₂ |
| 1-42 | O | H, H | H | H | H | S |
| 1-43 | H, H | O | H | H | H | S |
| 1-44 | H, H | O | H | H | CH═CHI | CH₂ |

⁽¹⁾R¹, R⁵, and R⁶ are each H except where noted.
⁽²⁾A¹ and A² are H, H; H, OH; or both are combined together to represent oxygen, where indicated.
⁽³⁾B¹ and B² are H, H; H, OH; or both are combined together to represent oxygen, where indicated.
⁽⁴⁾Compound 1-5 is a mixture of compounds in a 5/1 molar ratio where A¹A² = H, H; B¹B² = O/A¹A² = O; B¹B² = H, H.
⁽⁵⁾Compound 1-8 is a mixture of compounds in a 2/1 molar ratio where A¹A² = H, H; B¹B² = O/A¹A² = O; B¹B² = H, H.
⁽⁷⁾R⁶ = Br.
⁽⁸⁾R⁵ = Br.
⁽⁹⁾R⁶ = F
⁽¹⁰⁾R⁶ = 2-pyridylvinyl
⁽¹¹⁾R⁶ = 2-pyridylethyl
⁽¹²⁾2-pyr = 2-pyridyl

Preferred compounds of Table A include compounds 1-2, 1-3, 1-12, 1-17, 1-23, and 1-29.

Compounds having the formula listed in Table A may be formed by the methods of synthesis as described, e.g., in U.S. Pat. No. 5,705,511, which also show the ability of certain of these compounds to inhibit trkA tyrosine kinase activity, the disclosures of which are incorporated herein by reference in its entirety.

In another aspect of the present invention, the therapeutic composition includes an active agent having the one of the following structures, below.

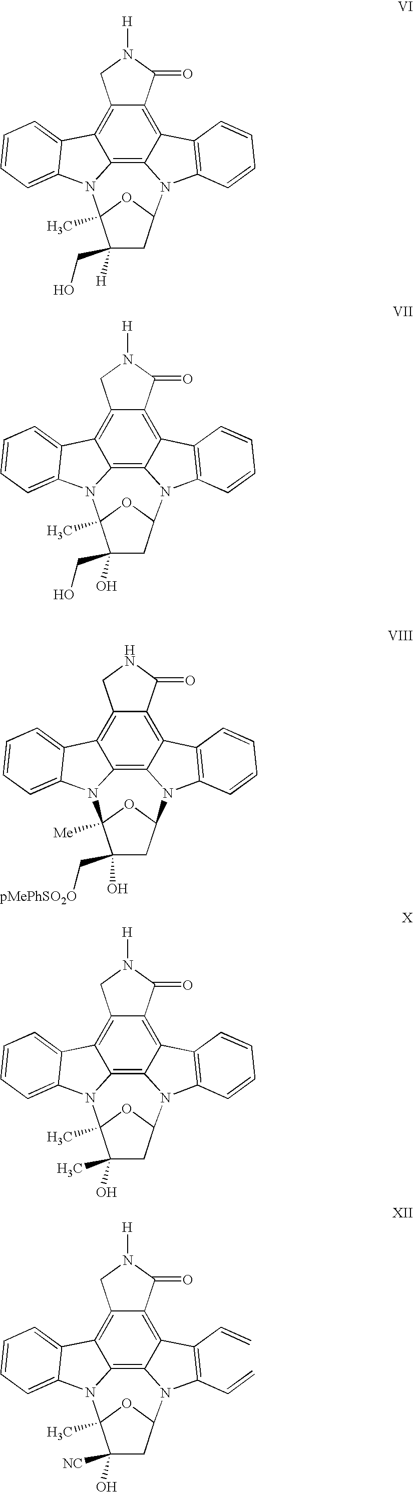

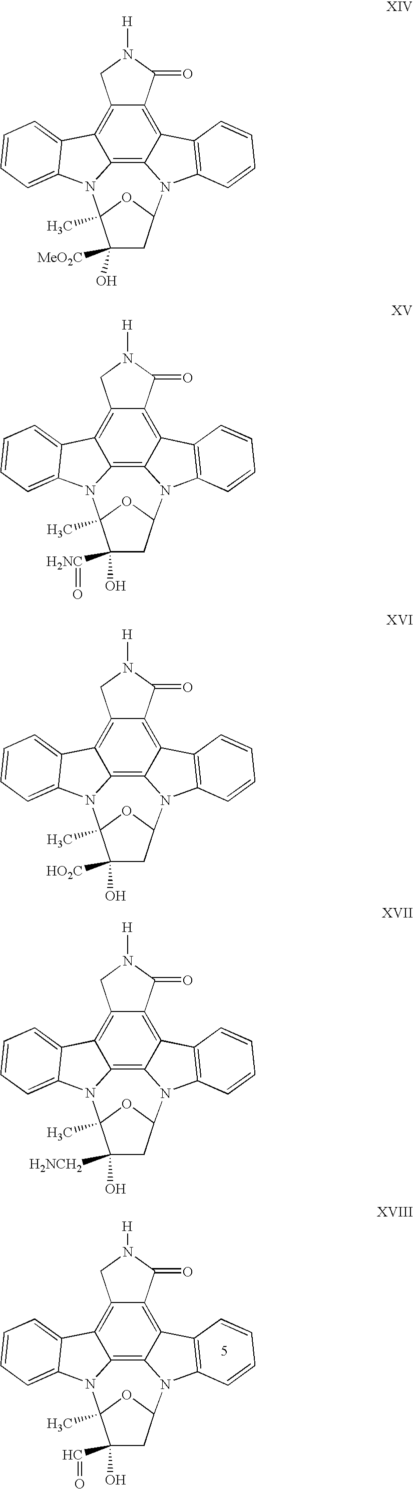

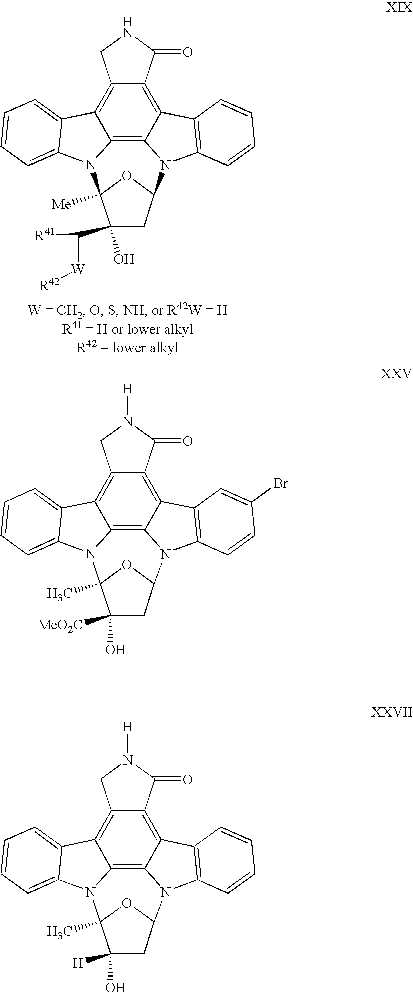

Preferred compounds have structures as depicted in VI, VII, X, XIV, or XV.

The compounds having the foregoing structures, VI, VII, VIII, X, XII, XIV, XV, XVI, XVII, XVIII, XIX, XXV and XXVII, may be formed by the methods of synthesis as described, e.g., in U.S. Pat. No. 6,093,713, which also shows the ability of certain of these compounds to inhibit trkA tyrosine kinase activity, the disclosures of which are incorporated herein by reference in its entirety.

In another aspect of the present invention, the therapeutic composition includes an active agent having the formula listed in Table B, below, and in accordance with Formula 2, which include that R¹, R⁴, R⁶, R⁷ are H; Y is O; and n is 1.

Formula 2:

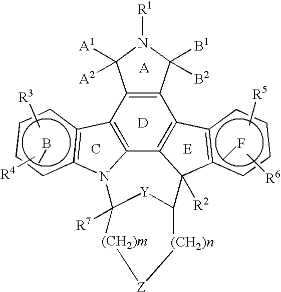

| Compound | | | | | | | | |
|---|---|---|---|---|---|---|---|---|
| No. | A₁A₂ | B₁B₂ | R₂ | R₃ | R₅ | R₈ | Z | m |
| 2-1 | H, H | O | H | H | H | H | bond | 1 |
| 2-1b | H, H | O | H | H | H | H | bond | 1 |
| 2-3 | H, H | O | H | H | H | Me | bond | 1 |
| 2-5 | H, H | O | H | 3-Br | H | Me | bond | 1 |
| 2-6 | H, H | O | H | H | 10-OMe | H | bond | 1 |
| 2-7a | H, H | O | H | H | H | Me | O | 1 |
| 2-7b | H, H | O | H | H | H | Me | O | 1 |
| 2-8 | O | H, H | H | H | H | H | bond | 1 |
| 2-9 | H, H | O | H | 3-(3′-NH₂-Ph) | H | H | bond | 1 |
| 2-11 | H, H | O | H | H | H | CO₂-Et | bond | 1 |
| 2-12 | H, H | O | H | H | H | CH₂—OH | bond | 1 |
| 2-13 | H, H | O | H | H | 9-0Me | H | bond | 1 |
| 2-14a | H, H | O | H | H | H | H | bond | 1 |
| 2-14b | H, H | O | H | H | H | H | bond | 1 |
| 2-15 | H, H | O | H | 3-CH₂O—CH₂OEt | H | H | bond | 1 |
| 2-16a | H, H | O | H | H | H | H | O | 1 |
| 2-16b | H, H | O | H | H | H | H | O | 1 |

Preferred compounds described in Table B include compounds with the formula 2-1, 2-3, 2-5, 2-6, 2-7a, 2-7b, 2-11, 2-12, and 2-14a.

Compounds having the formula listed in Table B may be formed by the methods of synthesis as described, e.g., in U.S. Pat. No. 6,127,401, which also shows the ability of certain of these compounds to inhibit trkA tyrosine kinase activity, the disclosures of which are incorporated herein by reference in its entirety.

In another aspect of the present invention, the therapeutic composition includes an active agent having the formula listed in Table C1 and C2, below.

In Table C1, the values correspond to the structure shown in Formula 3A, wherein the values for R1, R4, and R6 are H; Q is NH and G is a bond. In Table C2, the values correspond to the structure shown in Formula 3B, wherein the values for R1, R4, R5, R6, and R8 are H; W is CH₂, m is equal to 0 and G is CH₂.

TABLE C1
Formula 3A

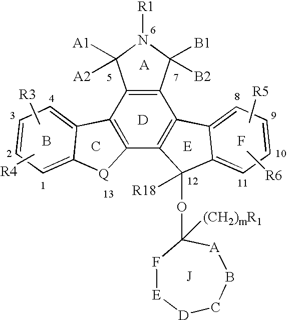

Com-

| pound | | | | | | | | | | | | | | |
|---|---|---|---|---|---|---|---|---|---|---|---|---|---|---|
| No. | A1A2 | B1B2 | R3 | R5 | R18 | m | R8 | A | B | C | D | E | F | Comments |
| 3a-02 | H2 | O | H | H | H | 0 | OH | CH2 | CH2 | N(Bn) | bond | CH2 | CH2 | |
| 3A-03 | H2 | O | H | H | H | 0 | OH | CH2 | CH2 | O | bond | CH2 | CH2 | |
| 3A-04 | H2 | O | H | H | H | 1 | H | O | CH2 | CH2 | CH2 | bond | bond | mixture of |
| | | | | | | | | | | | | | | diastereomers |
| 3A-05 | H2 | O | H | H | H | 0 | H | O | C(═O) | CH2 | CH2 | CH2 | bond | mixture of |
| | | | | | | | | | | | | | | diastereomers |
| 3A-06 | H2 | O | H | H | H | 0 | H | O | C(═O) | CH2 | CH2 | bond | bond | mixture of |
| | | | | | | | | | | | | | | diastereomers |
| 3A-07 | H2 | O | H | H | H | 0 | H | O | CH2 | CH2 | CH2 | bond | bond | mixture of |
| | | | | | | | | | | | | | | diastereomers |
| 3A-08 | H2 | O | H | H | H | 0 | (p)-F-phenyl | O | CH2 | CH2 | CH2 | bond | bond | mixture of |
| | | | | | | | | | | | | | | diastereomers |
| 3A-09 | H2 | O | H | H | H | 0 | 2-theinyl | O | CH2 | CH2 | CH2 | bond | bond | |
| 3A-10 | H2 | O | H | H | H | 0 | OH | CH2 | CH2 | N(Me) | bond | CH2 | CH2 | |
| 3A-11 | H2 | O | H | H | H | 0 | H | CH2 | S | CH2 | CH(OH) | bond | bond | mixture of |
| | | | | | | | | | | | | | | diastereomers |
| 3A-12 | H2 | O | H | H | H | 1 | H | O | CH2 | CH2 | CH2 | CH2 | bond | mixture of |
| | | | | | | | | | | | | | | diastereomers |
| 3A-13 | H2 | O | H | H | H | 0 | H | O | CH2 | CH2 | CH2 | CH2 | bond | mixture of |
| | | | | | | | | | | | | | | diastereomers |
| 3A-14 | H2 | O | H | H | H | 0 | OH | CH2 | CH2 | S | bond | CH2 | CH2 | |
| 3A-15 | H2 | O | H | H | H | 0 | OH | CH2 | 1,6-benzo- | | bond | CH2 | CH2 | mixture of |
| | | | | | | | | | fused | | | | | diastereomers |
| 3A-16 | H2 | O | H | H | H | 0 | OH | CH2 | N(Et) | CH2 | bond | CH2 | CH2 | mixture of |
| | | | | | | | | | | | | | | diastereomers |
| 3A-17 | H2 | O | H | H | H | 0 | OH | CH[CH2—N{(CH2)2}2O] | CH2 | bond | bond | CH2 | CH2 | mixture of |
| | | | | | | | | | | | | | | diastereomers |
| 3A-18 | H2 | O | H | H | H | 0 | OH | CH2 | CH2 | CH2 | bond | bond | bond | |
| 3A-19 | H2 | O | H | H | H | 3 | Cl | O | CH2 | CH2 | CH2 | bond | bond | mixture of |
| | | | | | | | | | | | | | | diastereomers |
| 3A-20 | H2 | O | H | H | H | 1 | O(C═O)CH2-t-Bu | O | CH2 | CH2 | CH2 | bond | bond | mixture of |
| | | | | | | | | | | | | | | diastereomers |
| 3A-21 | H2 | O | H | H | H | 1 | OH | O | CH2 | CH2 | CH2 | bond | bond | mixture of |
| | | | | | | | | | | | | | | diastereomers |
| 3A-22 | H2 | O | H | H | H | 1 | O(C═O)CH3 | O | CH2 | CH2 | CH2 | bond | bond | mixture of |
| | | | | | | | | | | | | | | diastereomers |
| 3A-23 | H2 | O | H | H | H | 0 | H | O | CH(OH) | CH2 | CH2 | bond | bond | mixture of |
| | | | | | | | | | | | | | | diastereomers |
| 3A-24 | H2 | O | H | H | H | 0 | OH | CH2 | CH2 | N[(C═O)CH3]) | bond | CH2 | CH2 | |
| 3A-25 | H2 | O | H | H | H | 1 | H | O | CH2 | —C(═CH2)— | CH2 | bond | bond | mixture of |
| | | | | | | | | | | | | | | diastereomers |
| 3A-26 | H2 | O | H | H | H | 1 | H | O | CH2 | —C[(OH) | CH2 | bond | bond | mixture of |
| | | | | | | | | | | (CH2OH)]— | | | | diastereomers |
| 3A-27 | H2 | O | H | H | H | 1 | H | O | CH2 | —C(═O)— | CH2 | bond | bond | mixture of |
| | | | | | | | | | | | | | | diastereomers |
| 3A-28 | H2 | O | H | H | H | 0 | —CH═CH2 | O | CH2 | CH2 | CH2 | bond | bond | mixture of |
| | | | | | | | | | | | | | | diastereomers |
| 3A-29 | H2 | O | H | H | H | 0 | OH | O | CH2 | CH2 | CH2 | bond | bond | mixture of |
| | | | | | | | | | | | | | | diastereomers |
| 3A-30a | H2 | O | H | H | H | 1 | H | O | CH2 | CH2 | CH2 | bond | bond | diastereomer A |
| 3A-30b | H2 | O | H | H | H | 1 | H | O | CH2 | CH2 | CH2 | bond | bond | diastereomer B |
| 3A-31 | H2 | O | H | H | H | 1 | —OCH2OCH2— | O | —C(═O)— | CH2 | CH2 | bond | bond | mixture of |
| | | | | | | | CH2OCH3 | | | | | | | diastereomers |
| 3A-32 | H2 | O | H | H | Et | 1 | —O(C═O)CH2- | O | CH2 | CH2 | CH2 | bond | bond | mixture of |
| | | | | | | | t-Bu | | | | | | | diastereomers |
| 3A-33 | H2 | O | H | H | H | 1 | OH | O | —C(═O)— | CH2 | CH2 | bond | bond | mixture of |
| | | | | | | | | | | | | | | diastereomers |
| 3A-34 | H2 | O | H | H | Et | 1 | OH | O | CH2 | CH2 | CH2 | bond | bond | mixture of |
| | | | | | | | | | | | | | | diastereomers |
| 3A-35 | H2 | O | H | H | H | 1 | OH | O | CH2 | CH2 | CH2 | bond | bond | diastereomer A |
| 3A-36 | H2 | O | H | H | H | 1 | OH | O | CH2 | CH2 | CH2 | bond | bond | diastereomer B |
| 3A-37 | O | H2 | H | H | H | 1 | H | O | CH2 | CH2 | CH2 | bond | bond | mixture of |
| | | | | | | | | | | | | | | diastereomers |
| 3A-38 | H2 | O | H | H | H | 0 | H | O | CH(OH) | CH2 | CH2 | bond | bond | single |
| | | | | | | | | | | | | | | diastereomer |
| 3A-40a | H2 | O | H | H | H | 0 | H | O | CH(OEt) | CH2 | CH2 | bond | bond | mixture of |
| | | | | | | | | | | | | | | diastereomers |
| | | | | | | | | | | | | | | AB |
| 3A-40b | H2 | O | H | H | H | 0 | H | O | CH(OEt) | CH2 | CH2 | bond | bond | mixture of |
| | | | | | | | | | | | | | | diastereomers |
| | | | | | | | | | | | | | | CD |
| 3A-42 | H2 | O | H | H | H | 0 | OH | O | CH2 | CH2 | CH2 | bond | bond | |
| 3A-43 | H2 | O | H | H | H | 0 | H | O | CH2 | CH2 | CH(OH) | bond | bond | mixture of |
| | | | | | | | | | | | | | | diastereomers |
| 3A-44 | H2 | O | H | H | H | 1 | Cl | O | CH2 | CH2 | CH2 | bond | bond | single |
| | | | | | | | | | | | | | | diastereomer |
| 3A-45a | H2 | O | H | H | H | 0 | H | O | 1,6-[2,4- | | CH2 | bond | bond | diastereomer A |
| | | | | | | | | | (OMe)2])- | | | | | |
| | | | | | | | | | benzo-fused | | | | | |
| 3A-45b | H2 | O | H | H | H | 0 | H | O | 1,6-[2,4- | | CH2 | bond | bond | diastereomer B |
| | | | | | | | | | (OMe)2])- | | | | | |
| | | | | | | | | | benzofused | | | | | |
| 3A-46 | H2 | O | H | H | Et | 0 | H | O | 1,6-[2,4- | | CH2 | bond | bond | diastereomer |
| | | | | | | | | | (OMe)2])- | | | | | |
| | | | | | | | | | benzofused | | | | | |
| 3A-47 | H2 | O | H | H | H | 0 | OH | C(═O) | O | CH2 | —C[(CH3)2]— | bond | bond | single |
| | | | | | | | | | | | | | | diastereomer |
| 3A-48 | H2 | O | H | H | H | 0 | OH | O | —CH[O(CMe2)O] | O | CH2 | bond | bond | mixture of |
| | | | | | | | | | CH— | | | | | diastereomers |

| | | | | | | | | | | | | |
|---|---|---|---|---|---|---|---|---|---|---|---|---|
| 3A-49 | H2 | O | H | H | H | 0 | -C═N- | NH | C(═O) | CH2 | CH2 | bond |

| | | | | | | | | | | | | | | |
|---|---|---|---|---|---|---|---|---|---|---|---|---|---|---|
| 3A-50 | H2 | O | H | H | H | 0 | OH | CH2 | CH2 | CH2 | CH2 | CH2 | bond | |
| 3A-51a | H2 | O | H | H | H | 1 | H | O | CH(OEt) | CH2 | O | CH2 | bond | diastereomer A |
| 3A-51bc | H2 | O | H | H | H | 1 | H | O | CH(OEt) | CH2 | O | CH2 | bond | diastereomers |
| | | | | | | | | | | | | | | B & C |
| 3A-51d | H2 | O | H | H | H | 1 | H | O | CH(OEt) | CH2 | O | CH2 | bond | diastereomer D |
| 3A-52 | H2 | O | 3- | H | H | 0 | H | O | CH(OCH2— | CH2 | CH2 | bond | bond | mixture of |
| | | | (C═O ) | | | | | | CH2OCH3) | | | | | |
| | | | O— | | | | | | | | | | | |
| | | | OCH3 | | | | | | | | | | | |
| 3A-53 | H2 | O | H | 10- | H | 1 | OH | O | CH2 | CH2 | CH2 | bond | bond | single |
| | | | | O-Me | | | | | | | | | | diastereomer |
| 3A-54 | H2 | O | H | 10- | H | 1 | OH | O | CH(OEt) | CH2 | CH2 | bond | bond | single |
| | | | | O-Me | | | | | | | | | | diastereomer |
| 3A-55 | H2 | O | H | H | H | 0 | H | CH(COOEt) | C(═O) | CH2 | CH2 | bond | bond | single |
| | | | | | | | | | | | | | | diastereomer |
| 3A-56 | O | O | H | H | H | 0 | H | CH(COOEt) | C(═O) | CH2 | CH2 | bond | single | |
| | | | | | | | | | | | | | | diastereomer |
| 3A-59 | H2 | O | H | H | H | O | H | CH2 | CH2 | CH2 | CH2 | bond | bond | single |
| | | | | | | | | | | | | | | diastereomer |
| 3A-60 | H2 | O | H | H | H | O | H | C(═0) | O | CH2 | CH2 | bond | bond | single |
| | | | | | | | | | | | | | | diastereomer |

TABLE C2
Formula 3B

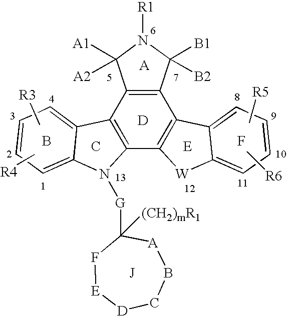

| Compound | | | | | | | | | | |
|---|---|---|---|---|---|---|---|---|---|---|
| No. | A1A2 | B1B2 | R3 | A | B | C | D | E | F | Comments |
| 3B-01a | H2 | O | H | O | CH2 | bond | bond | bond | bond | racemate |
| 3B-01c | H2 | O | H | O | CH2 | bond | bond | bond | bond | (S) |
| | | | | | | | | | | enantiomer |
| 3B-01b | H2 | O | H | O | CH2 | bond | bond | bond | bond | (R) |
| | | | | | | | | | | enantiomer |
| 3B-39 | H2 | O | H | C(═O) | CH2 | bond | bond | bond | bond | |
| 3B-41 | H2 | O | H | C(OH) | CH2 | CH2 | bond | bond | bond | mixture of |
| | | | | | | | | | | diastereomers |
| 3B-57 | H2 | O | 3-Br | O | CH2 | bond | bond | bond | bond | racemate |
| 3B-58 | H2 | O | 3-CH2OCH2— | O | CH2 | bond | bond | bond | bond | racemate |
| | | | CH3 | | | | | | | |
| 3B-61 | H2 | O | 3-CH2OCH2— | O | CH2 | bond | bond | bond | bond | racemate |
| | | | CH2OCH3 | | | | | | | |
| 3B-62 | H2 | O | H | O | CH2 | CH2 | CH2 | CH2 | bond | racemate |
| 3B-63 | H2 | O | H | CH2 | O | CH2 | CH2 | CH2 | bond | racemate |

Preferred compounds described in Table C1 and C2 include compounds with the formula 3B-01a, 3A-02, 3A-03, 3A-04, 3A-06, 3A-07, 3A-21, 3A-22, 3A-23, 3A-26, 3A-29, 3A-35, 3A-36, 3A-40a, 3A-40b, 3A-44, 3A-47, 3A-52, 3A-53, and 3A-54.

Compounds having the formula listed in Table C1 and C2 may be formed by the methods of synthesis as described, e.g., in PCT Publ. No. WO 00/47583, which also shows the ability of certain of these compounds to inhibit trkA tyrosine kinase activity, the disclosures of which are incorporated herein by reference in its entirety.

Table D1 and D2, below, lists a number of compounds by defining the substituents X, R, R¹ and R² (see footnotes) according to Formula 4:

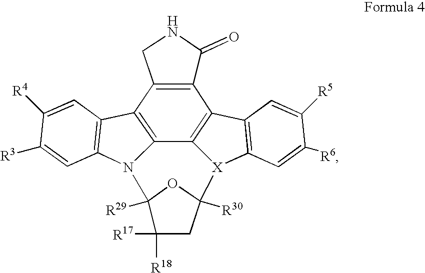

which are useful for treating proliferative skin disorders, preferably psoriasis, in accordance with the present invention; wherein R³, R⁴, R⁶, and R³⁰ are H, R²⁹ is methyl and X is CH.

TABLE D1

| Compound⁽¹⁾ | R¹⁸ | R¹⁷ | R⁵ |
|---|---|---|---|
| 1 | CO₂CH₃ | OH | H |
| 2 | CH₂OH | OH | H |
| 3 | H | OH | H |
| 4 | CONH₂ | OH | H |
| 5 | CO₂CH₃ | OH | OH |
| 6 | CH₂OCOCH₃ | OH | H |
| 7⁽²⁾ | —CH₂NHCO₂— | — | H |
| 8 | CH₂SOCH₃ | OH | H |
| 9 | CONHC₂H₅ | OH | H |
| 10 | CONHC₃H₇ | OH | H |
| 11 | 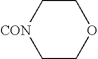 | OH | H |
| 12 | CONH(CH₂)₂OH | OH | H |
| 13 | —CH₂OC(CH₃)₂O— | — | H |
| 14 | CH═NNHCONH₂ | OH | H |
| 15⁽²⁾ | —CH₂N(CH₃)CO₂— | — | H |
| 16 | CH₂N(CH₃)₂ | OH | H |
| 17 | CH₂NH-Pro | OH | H |
| 18⁽³⁾ | CH₂NH-Ser | OH | H |
| 19 | CH₂OH | OCH₃ | H |
| 20⁽⁴⁾ | CH₂S-Glc | OH | H |
| 21 | CH₂N₃ | OH | H |
| 23 | CO₂CH₃ | OH | Br |
| 24 | CH₂NHCOCH₃ | OH | H |
| 25 | CON(CH₃)₂ | OH | H |
| 26 | CONHOH | OH | H |
| 27 | CO₂CH₃ | OH | NHCONHC₆H₅ |
| 28 | CH═NOH | OH | H |
| 29 | CH=NNHC(═NH)—NH₂ | OH | H |
| 30 | 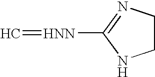 | OH | H |
| 31 | CH₂CO₂CH₃ | OH | H |
| 32^{(3,7)} | CH₂NH-Gly | OH | H |
| 33 | CONHC_{H}₅ | OH | H |
| 34 | CO₂CH₃ | OH | NHCONHC₆H₅ |
| 35 | CO₂CH₃ | OH | CH₂OCONIHC₂H₅ |
| 36 | CH₂OH | OH | Br |
| 37 | CO₂CH₃ | OH | NHCO₂CH₃ |
| 38 | CO₂CH₃ | OH | CH₃ |
| 39⁽⁵⁾ | CO₂CH₃ | OH | Br |
| 40 | 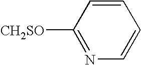 | OH | H |
| 41 | CO₂CH₃ | OH | CH²OC₂H₅ |
| 42⁽⁵⁾ | CH₂OH | OH | Br |
| 43⁽⁵⁾ | CONHCH₂CH₂OH | OH | Br |
| 44⁽⁶⁾ | CO₂CH₃ | OH | Cl |
| 45 | CONH₂ | OH | Br |
| 46 | CH₂NHCONHC₂H₅ | OH | H |
| 47 | CH₂NHCONHC₆H₅ | OH | H |
| 48 | CH═NN(C₆H₅)₂ | OH | H |
| 49 | CH₂SC₆H₅ | OH | H |
| 50 | 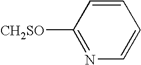 | OH | H |
| 51 | CH₂SOC₆H₅ | OH | H |

⁽¹⁾R² is hydrogen except where noted in footnotes 6, 7, and 8.
⁽²⁾X and R are combined together to form the linking group.
⁽³⁾NH-amino acid linkage is an amide bond through the carboxyl group of the amino acid.
⁽⁴⁾Glc is glucose; linkage is through the 1-position.
⁽⁵⁾R² is Br.
⁽⁶⁾R² is Cl.
⁽⁷⁾Compound is in the form of the hydrochloride.

TABLE D2

| Compound⁽¹⁾ | X | R | R¹ |
|---|---|---|---|
| 52 | ═O | | H |
| 53 | CO₂CH₃ | OCH₃ | H |
| 54 | CONHCH₃ | OH | H |
| 55 | CONH(i-Butyl) | OCOCH₃ | H |
| 56 | CH₂SCH₃ | OH | H |
| 57⁽³⁾ | CH₂NH-Lys | OH | H |
| 58 | CO₂CH₃ | OH | CH(SC₆H₅)₂ |
| 59 | CO₂CH₃ | OH | CH(—SCH₂CH₂S—) |
| 60 | CO₂CH₃ | OH | 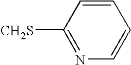 |
| 61 | CO₂CH₃ | OH | 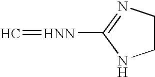 |
| 62 | CO₂CH₃ | OH | 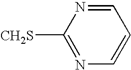 |
| 63 | CO₂CH₃ | OH | 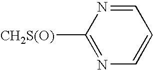 |
| 64 | CO₂CH₃ | OH | 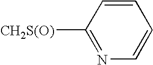 |
| 65⁽⁴⁾ | CO₂CH₃ | OH | CH₂SC₂H₅ |
| 66⁽⁵⁾ | CO₂CH₃ | OH | CH₂S(O)C₂H₅ |
| 67 | CO₂CH₃ | OH | 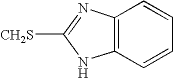 |
| 68 | CO₂CH₃ | OH | 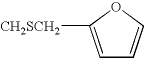 |
| 69 | CO₂CH₃ | OH | 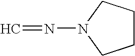 |
| 70 | CO₂CH₃ | OH | 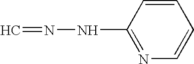 |
| 71 | CO₂CH₃ | OH | CH₂SCH₂CH₂N(CH₃)₂ |
| 72 | CO₂CH₃ | OH | 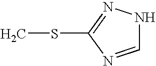 |
| 73 | CO₂CH₃ | OH | CH═NNHC(═NH)NH₂ |
| 74 | CO₂CH₃ | OH | 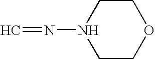 |
| 75 | CO₂CH₃ | OH | CH═N—N(CH₃)₂ |
| 76 | CO₂CH₃ | OH | 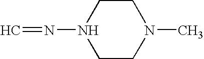 |

⁽¹⁾R² is hydrogen except where noted in footnotes 4 and 5.
⁽²⁾X and R are combined together to form the linking group.
⁽³⁾NH-amino acid linkage is an amide bond through the carboxyl group of the amino acid.
⁽⁴⁾R² is CH2OH.
⁽⁵⁾R² is CH₂S(O)C₂H₅.

Compounds having the formula listed in Table D1 and D2, in particular compound 2 of Table D1, may be formed by the methods of synthesis as described, e.g., in Lewis et. al. (U.S. Pat. No. 5,756,494), the disclosures of which are incorporated herein by reference in its entirety.

Definitions

The term “treatment” means to therapeutically improve and/or reduce and/or make more therapeutically tolerable, or eliminate the symptoms or cause of a disease or clinical condition of that disease, in order to improve on the quality of life of an afflicted person. The term treatment is meant to include “alleviate,” “ameliorate” or “ameliorating,” which means to therapeutically improve and/or reduce and/or make more therapeutically tolerable in order to improve on the quality of life of an afflicted person.

The acronym “NGF” refers to nerve growth factor.

The terms “inhibit” or “inhibiting” in reference to NGF or NGF-receptors mean that the presence of the compounds of the present invention have a comparatively greater effect on reducing and/or prohibiting and/or preventing the binding of NGF to its natural receptors, either p75 or trkA, and prevent the downstream signaling effects, including mitogenic activity and protection from apoptosis.

The term “NGF receptor” refers to the natural receptors of NGF, which include p75 or trkA.

As used herein, the term “Atrk” refers to the family of high affinity neurotrophin receptors presently comprising trk A, trk B, and trk C, and other membrane associated proteins to which a neurotrophin, particularly NGF can bind.

The terms “Pro” “Ser” “Gly” “Lys” refer to the three letter abbreviation for amino acids.

The term “alkyl” means a straight-chain, cyclic, or branched alkyl group having 1 to 8 carbon atoms, such as methyl, ethyl, propyl, isopropyl, butyl, isobutyl, sec-butyl, tert-butyl, pentyl, isoamyl, neopentyl, 1-ethylpropyl, hexyl, octyl, cyclopropyl, and cyclopentyl. The alkyl moiety of alkyl-containing groups, such as alkoxy, alkoxycarbonyl, and alkylaminocarbonyl groups, has the same meaning as alkyl defined above. Cyclic alkyl groups are also referred to as “cycloalkyl.” Lower alkyl groups, which are preferred, are alkyl groups as defined above which contain 1 to 4 carbons. The term “alkenyl” is intended to include straight-chain or branched hydrocarbon chains having at least one carbon-carbon double bond. Examples of alkenyl groups include ethenyl and propenyl groups. As used herein, the term “alkynyl” is intended to include straight-chain or branched hydrocarbon chains having at least one carbon-carbon triple bond. Examples of alkynyl groups include ethynyl and propynyl groups. A designation such as “alkyl of 1-4 carbons” or “alkyl having 1 to 4 carbons” refers to an alkyl group containing from 1 to 4 carbon atoms.

The term “alkylene” means an alkane, preferably a linear alkane, with two hydrogen atoms removed, e.g., methylene (—CH₂—), or ═CH₂.

A “heterocycloalkyl” is a cyclic alkyl of 3 to 7 carbon atoms in which one or more ring carbon atom is replaced by a hetero (i.e., non-carbon) atom such as O, N or S.

The acyl moiety of acyl-containing groups such as acyloxy groups is intended to include a straight-chain or branched alkanoyl group having 1 to 6 carbon atoms, such as formyl, acetyl, propanoyl, butyryl, valeryl, pivaloyl or hexanoyl.

One type of alkyl is an “O-n-alkyl,” which means a group that is a straight chain alkyl with an oxygen atom at one of the terminal ends. When bound to another carbon at the oxygen atom, the result is the formation of an ether moiety.

As used herein, the term “aryl” means a group having 6 to 12 carbon atoms such as phenyl, biphenyl and naphthyl. Preferred aryl groups include unsubstituted or substituted phenyl and naphthyl groups. The term “heteroaryl” as used herein denotes an aryl group in which one or more ring carbon atom is replaced by a hetero (i.e., non-carbon) atom such as O, N or S. Preferred heteroaryl groups include pyridyl, pyrimidyl, pyrrolyl, furyl, thienyl, imidazolyl, triazolyl, tetrazolyl, quinolyl, isoquinolyl, benzoimidazolyl, thiazolyl, pyrazolyl, and benzothiazolyl groups.

The term “aralkyl” (or “arylalkyl”) is intended to denotes a group having from 7 to 15 carbons, consisting of an alkyl group that bears an aryl group. Examples of aralkyl groups include benzyl, phenethyl, benzhydryl and naphthylmethyl groups. Alkyl groups and alkyl moieties contained within substituent groups such as aralkyl, alkoxy, arylalkoxy, hydroxyalkoxy, alkoxy-alkoxy, hydroxy-alkylthio, alkoxy-alkylthio, alkylcarbonyloxy, hydroxyalkyl and acyloxy groups may be substituted or unsubstituted. A substituted alkyl group has 1 to 3 independently-selected substituents, preferably hydroxy, lower alkoxy, lower alkoxy-alkoxy, substituted or unsubstituted arylalkoxy-lower alkoxy, substituted or unsubstituted heteroarylalkoxy-lower alkoxy, substituted or unsubstituted arylalkoxy, substituted or unsubstituted heterocycloalkoxy, halogen, carboxyl, lower alkoxycarbonyl, nitro, amino, mono- or di-lower alkylamino, dioxolane, dioxane, dithiolane, dithione, furan, lactone, or lactam.

Heterocyclic groups or “heterocycloalkyls” formed with a nitrogen atom include pyrrolidinyl, piperidinyl, piperidino, morpholinyl, morpholino, thiomorpholino, N-methylpiperazinyl, indolyl, isoindolyl, imidazole, imidazoline, oxazoline, oxazole, triazole, thiazoline, thiazole, pyrazole, pyrazolone, and triazole groups. Heterocyclic groups formed with an oxygen atom includes furan, tetrahydrofuran, pyran, and tetrahydropyran groups.

“Hydroxyalkyl” groups are alkyl groups that have a hydroxyl group appended thereto. Halogens include fluorine, chlorine, bromine and iodine.

As used herein, the term “heteroarylalkyl” means an arylalkyl group that contains a heteroatom. The term “oxy” denotes the presence of an oxygen atom. Thus, “alkoxy” groups are alkyl groups that are attached through an oxygen atom, and “carbonyloxy” groups are carbonyl groups that are attached through an oxygen atom.

The term “heterocycloalkoxy” means an alkoxy group that has a heterocyclo group attached to the alkyl moiety thereof, and the term “arylalkoxy” means an alkoxy group that has an aryl group attached to the alkyl moiety thereof. The term “alkylcarbonyloxy” means an group of formula —O—C(═O)-alkyl.

The term “alkylcarbonyloxy” means an alkyl group having a carbonyl, or acyl, functional group with a terminal reactive oxygen.

As used herein, the term “alkyloxy-alkoxy” denotes an alkoxy group that contains an alkyloxy substituent attached to its alkyl moiety. The term “alkoxy-alkylthio” means an alkylthio group (i.e., a group of formula —S-alkyl) that contains an alkoxy substituent attached to its alkyl moiety. The term “hydroxy-alkylthio” means an alkylthio group (i.e., a group of formula —S-alkyl) that contains a hydroxy substituent attached to its alkyl moiety.

As used herein, the term “monosaccharide” has its accustomed meaning as a simple sugar.

As used herein, the term “amino acid” denotes a molecule containing both an amino group and a carboxyl group. Embodiments of amino acids include α-amino acids; i.e., carboxylic acids of general formula HOOC—CH(NH₂)-(side chain).

Side chains of amino acids include naturally occurring and non-naturally occurring moieties. Non-naturally occurring (i.e., unnatural) amino acid side chains are moieties that are used in place of naturally occurring amino acid side chains in, for example, amino acid analogs. See, for example, Lehninger, Biochemistry, Second Edition, Worth Publishers, Inc, 1975, pages 73-75, incorporated by reference herein.

Preferred α-amino acids include glycine, alanine, proline, glutamic acid, and lysine, having the D configuration, the L configuration, or as a racemate.

The side chains of further representative α-amino acids are shown below in Table i:

TABLE i

| | |
|---|---|
| H | HS—CH₂— |
| CH₃— | HO₂C—CH(NH₂)—CH₂—S—S—CH₂— |
| HO—CH₂— | CH₃—CH₂— |
| C₆H₅—CH₂— | CH₃—S—CH₂—CH₂— |
| HO—C₆H₄—CH₂— | CH₃—CH₂—S—CH₂—CH₂— |
| | HO—CH₂—CH₂— |
| 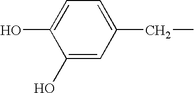 | C₅H₉—C₆H₁₁—C₆H₁₁—CH₂— |
| 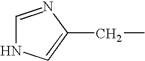 | CH₃—CH(OH)—HO₂C—CH₂—NHC(═O)—CH₂—HO₂C—CH₂— |
| 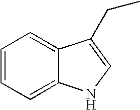 | HO₂C—CH₂—CH₂—NH₂C(═O)—CH₂—NH₂C(═O)—CH₂—CH₂— |
| 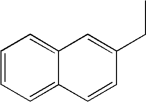 | (CH₃)₂—CH—(CH₃)₂—CH—CH₂—CH₃—CH₂—CH₂—H₂N—CH₂—CH₂—CH₂— |
| 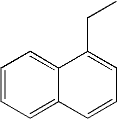 | H₂—C(═NH)—NH—CH₂—CH₂—CH₂—H₂N—C(═O)—NH—CH₂—CH₂—CH₂—CH₃—CH₂—CH(CH₃)—CH₃—CH₂—CH—CH₂—H₂N—CH₂—CH₂—CH₂—CH₂— |

The term “Glc” refers to glucose.

The term “furan” is used herein to refer to a furyl substituent. “Position 2” or “2 position” of a furan refers to standard nomenclature in the art specifying a particular ring carbon of the furan. Additionally, a “linking furan” is a furan group that links two atoms of another ring structure, e.g., a pyrrolocarbazole, especially at the 12 and 13 position. The term “linking furan via its 2 and 5 position” indicates the positions on the linking furan that link the furan to another ring structure, e.g., a pyrrolocarbazole.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, the preferred methods and materials are described below. All publications, patent applications, patents, and other references mentioned herein are incorporated by reference in their entirety. In case of conflict, the present document, including definitions, will control. Unless otherwise indicated, materials, methods, and examples described herein are illustrative only and not intended to be limiting. Various features and advantages of the invention will be apparent from the following detailed description and from the claims.

The compounds of the invention can be in the form of pharmaceutically acceptable salts including pharmaceutically acceptable acid addition salts, metal salts, ammonium salts, organic amine addition salts, and amino acid addition salts. Examples of the pharmaceutically acceptable acid addition salts are inorganic acid addition salts such as hydrochloride, sulfate, and phosphate; and organic acid addition salts such as acetate, maleate, fumarate, tartrate, and citrate. Examples of the pharmaceutically acceptable metal salts are alkali metal salts such as sodium salt and potassium salt, alkaline earth metal salts such as magnesium salt and calcium salt, aluminum salt, and zinc salt. Examples of the pharmaceutically acceptable ammonium salts are ammonium salt and tetraethyl ammonium salt. Examples of the pharmaceutically acceptable organic amine addition salts are salts with morpholine and piperidine. Examples of the pharmaceutically acceptable amino acid addition salts are salts with lysine, glycine, and phenylalanine.

Dosage and Formulation

For therapeutic purposes, the compounds of the present invention can be administered by any means that results in the contact of the active agent with the agent's site of action in the body of the subject. The compounds may be administered by any conventional means available for use in conjunction with pharmaceuticals, either as individual therapeutic agents or in combination with other therapeutic agents, such as, for example, analgesics. The compounds of the present invention are preferably administered in therapeutically effective amounts for the treatment of the diseases and disorders described herein to a subject in need thereof.

A therapeutically effective amount can be readily determined by the attending diagnostician, as one skilled in the art, by the use of conventional techniques. The effective dose will vary depending upon a number of factors, including the type and extent of progression of the disease or disorder, the overall health status of the particular patient, the relative biological efficacy of the compound selected, the formulation of the active agent with appropriate excipients, and the route of administration. Typically, the compounds are administered at lower dosage levels, with a gradual increase until the desired effect is achieved.

Typical dose ranges are from about 0.01 mg/kg to about 100 mg/kg of body weight per day, with a preferred dose from about 0.01 mg/kg to 10 mg/kg of body weight per day. A preferred daily dose for adult humans includes about 25, 50, 100 and 200 mg, and an equivalent dose in a human child. The compounds may be administered in one or more unit dose forms. The unit dose ranges from about 1 to about 500 mg administered one to four times a day, preferably from about 10 mg to about 300 mg, two times a day. In an alternate method of describing an effective dose, an oral unit dose is one that is necessary to achieve a blood serum level of about 0.05 to 20 μg/ml in a subject, and preferably about 1 to 20 μg/ml.

The compounds of the present invention may be formulated into pharmaceutical compositions by admixture with one or more pharmaceutically acceptable excipients. The excipients are selected on the basis of the chosen route of administration and standard pharmaceutical practice, as described, for example, in Remington: The Science and Practice of Pharmacy, 20^{th} ed.; Gennaro, A. R., Ed.; Lippincott Williams & Wilkins: Philadelphia, Pa., 2000. The compositions may be formulated to control and/or delay the release of the active agent(s), as in fast-dissolve, modified-release, or sustained-release formulations. Such controlled-release, or extended-release compositions may utilize, for example biocompatible, biodegradable lactide polymers, lactide/glycolide copolymers, polyoxyethylene-polyoxypropylene copolymers, or other solid or semisolid polymeric matrices known in the art.

The compositions can be prepared for administration by oral means; parenteral means, including intravenous, intramuscular, and subcutaneous routes; topical or transdermal means; transmucosal means, including rectal, vaginal, sublingual and buccal routes; ophthalmic means; or inhalation means. Preferably the compositions are prepared for oral administration, particularly in the form of tablets, capsules or syrups; for parenteral administration, particularly in the form of liquid solutions, suspensions or emulsions; for intranasal administration, particularly in the form of powders, nasal drops, or aerosols; or for topical administration, such as creams, ointments, solutions, suspensions aerosols, powders and the like.

For oral administration, the tablets, pills, powders, capsules, troches and the like can contain one or more of the following: diluents or fillers such as starch, or cellulose; binders such as microcrystalline cellulose, gelatins, or polyvinylpyrrolidones; disintegrants such as starch or cellulose derivatives; lubricants such as talc or magnesium stearate; glidants such as colloidal silicon dioxide; sweetening agents such as sucrose or saccharin; or flavoring agents such as peppermint or cherry flavoring. Capsules may contain any of the afore listed excipients, and may additionally contain a semi-solid or liquid carrier, such as a polyethylene glycol. The solid oral dosage forms may have coatings of sugar, shellac, or enteric agents. Liquid preparations may be in the form of aqueous or oily suspensions, solutions, emulsions, syrups, elixirs, etc., or may be presented as a dry product for reconstitution with water or other suitable vehicle before use. Such liquid preparations may contain conventional additives such as surfactants, suspending agents, emulsifying agents, diluents, sweetening and flavoring agents, dyes and preservatives.

The compositions may also be administered parenterally. The pharmaceutical forms acceptable for injectable use include, for example, sterile aqueous solutions, or suspensions. Aqueous carriers include mixtures of alcohols and water, buffered media, and the like. Nonaqueous solvents include alcohols and glycols, such as ethanol, and polyethylene glycols; oils, such as vegetable oils; fatty acids and fatty acid esters, and the like. Other components can be added including surfactants; such as hydroxypropylcellulose; isotonic agents, such as sodium chloride; fluid and nutrient replenishers; electrolyte replenishers; agents which control the release of the active compounds, such as aluminum monostearate, and various co-polymers; antibacterial agents, such as chlorobutanol, or phenol; buffers, and the like. The parenteral preparations can be enclosed in ampules, disposable syringes or multiple dose vials. Other potentially useful parenteral delivery systems for the active compounds include ethylene-vinyl acetate copolymer particles, osmotic pumps, implantable infusion systems, and liposomes.

Other possible modes of administration include formulations for inhalation, which include such means as dry powder, aerosol, or drops. They may be aqueous solutions containing, for example, polyoxyethylene-9-lauryl ether, glycocholate and deoxycholate, or oily solutions for administration in the form of nasal drops, or as a gel to be applied intranasally. Formulations for topical use are in the form of an ointment, cream, or gel. Typically these forms include a carrier, such as petrolatum, lanolin, stearyl alcohol, polyethylene glycols, or their combinations, and either an emulsifying agent, such as sodium lauryl sulfate, or a gelling agent, such as tragacanth. Formulations suitable for transdermal administration can be presented as discrete patches, as in a reservoir or microreservoir system, adhesive diffusion-controlled system or a matrix dispersion-type system. Formulations for buccal administration include, for example lozenges or pastilles and may also include a flavored base, such as sucrose or acacia, and other excipients such as glycocholate. Formulations suitable for rectal administration are preferably presented as unit-dose suppositories, with a solid based carrier, such as cocoa butter, and may include a salicylate.

The compounds of the present invention can be employed as the sole active agent in a pharmaceutical or can be used in combination with other active ingredients, e.g., other NGF inhibitors or psoriasis treatments.

Psoriasis is a disorder of the skin characterized by hyperproliferation of keratinocytes within the epidermis. NGF is produced and secreted by human keratinocytes and in an elevated amount in areas of psoriatic lesions and trauma. The materials of the present invention provide inhibitors of the mitogenic affect of NGF, in particular the downstream affects of NGF binding to its receptors, and their use in accordance with the methods of the present invention. Furthermore, these materials also are useful for inhibiting proliferative skin diseases, psoriasis in particular, and for alleviating the symptoms associated with proliferative skin diseases in accordance with the present invention.
Most clinical trials for psoriasis utilize the Psoriasis Area and Severity Index (PASI) for quantitation, where a 75% reduction (PASI 75) is the benchmark for primary endpoint for the trial. Some in the field report that a 50% reduction (PASI 50) results in significant improvements and provides for significant upgrades in the quality of life of affected patients.
A patient undergoing treatment is analyzed by a dermatologist or an experienced physician for each area of the body that is afflicted with psoriasis. Each area of the body is rated for erythema, scaling, and thickness for the average plaque or the overall condition of plaques in that region. The plaques are rated as they are actually seen on the day of examination and not in comparison with baseline condition.

PASI Scoring

PASI scores can range from 0 to 72. Dermatologic disease severity is scored as follows:

Four main body areas will be assessed, the head, the trunk, the upper extremities, and the lower extremities corresponding to 10%, 30%, 20%, and 40% of the total body surface area (BSA), respectively. The area of psoriatic involvement for each body area will be assigned a numerical value according to degree of involvement as follows:
0=no involvement
1=<10% involvement
2=10% to <30% involvement
3=30% to <50% involvement
4=50% to <70% involvement
5=70% to <90% involvement
6=90% to 100% involvement

Conventions for estimating BSA include the following:
Include only currently active disease in affected area
For small, scattered lesions do not include the skin between the lesions in the estimate
For a centrally cleared plaque, count only area of inflamed outer ring
Do not include residual hyperpigmentation, hypopigmentation, pigmented macules, or diffuse slight pink coloration
Neck—include with the head
Buttocks—include with the lower extremities
Axillae—include with trunk
Genitals—include with the trunk
Separation of trunk and lower extremities—the inguinal canal separates the trunk and legs anteriorly

In addition, the severity of the psoriatic lesions in three main signs—erythema (E), thickness (T), and scaling (S)—will be assessed for each body area according to a scale (0-4) in which 0 represents a complete lack of cutaneous involvement and 4 represents the most severe possible involvement.

Psoriatic Lesion Signs

| 1. | | 2. Erythema ^{a} | 3. Scaling | 4. Thickness |
|---|---|---|---|---|
| 1) | 0 = none | No redness | No scaling | No elevation over |
| | | | | normal skin |
| 2) | 1 = slight | Faint redness | Fine scale partially | Slight but definite |
| | | | covering lesions | elevation, typically |
| | | | | edges indistinct or |
| | | | | sloped |
| 3) | 2 = moderate | Red coloration | Fine to coarse scale | Moderate elevation with |
| | | | covering most of all | rough or sloped edges |
| | | | of the lesions | |
| 4) | 3 = severe | Very or bright | Coarse, non-tenacious | Marked elevation |
| | | red coloration | scale predominates | typically with hard |
| | | | covering most or all | or sharp edges |
| | | | of the lesions | |
| 5) | 4 = very severe | Extreme red | Coarse, thick, tenacious | Very marked elevation |
| | | coloration; dusky | scale over most or all | typically with hard |
| | | to deep red | lesions; rough surface | sharp edges |
| | | coloration | | |

^{a} Do not include residual hyperpigmentation, hypopigmentation, pigmented macules, or diffuse slight pink coloration as erythema.

Calculating PASI

To calculate the PASI, the sum of the severity rating for the three main signs will be multiplied with the numerical value of the area affected and with the various percentages of the four body areas. These values will then be added to complete the formula as follows:
PASI=0.1(Eh+Th+Sh)Ah+0.3(Et+Tt+St)At+0.2(Eu+Tu+Su)Au+0.4(El+Tl+Sl)Al

| | | 5. | 6. | 7. Upper | 8. Lower |
|---|---|---|---|---|---|
| Row | 6) | Head | Trunk | Limbs | Limbs |
| 1 | 7) Erythema ^{a} | 8) | 9) | 10) | 11) |
| 2 | 12) Thickness ^{a} | | | | |
| 3 | 13) Scaling ^{a} | | | | |
| 4 | 14) Total each | | | | |
| | column | | | | |
| 5 | 15) Degree of | | | | |
| | involvement ^{b} | | | | |
| 6 | 16) Multiply | | | | |
| | Row 4 by Row 5 | | | | |
| 7 | 17) | ×0.10 | ×0.30 | ×0.20 | ×0.40 |
| 8 | 18) Multiply | | | | |
| | Row 6 by Row 7 | | | | |

| | |
|---|---|
| 9 | 19) Total PASI (add together |
| | each column from Row 8) |

^{a} Rank severity of psoriatic lesions: 0 = none, 1 = slight, 2 = moderate, 3 = severe, 4 = very severe.
^{b} Rank area of psoriatic involvement: 0 = none, 1 = <10%, 2 = 10% to <30%, 3 = 30% to <50%, 4 = 50% to <70%, 5 = 70% to <90%, 6 = 90% to 100%.

A PASI score is determined for each patient at study baseline and at final study assessment. The numbers of patients who meet the criteria for PASI-50 and PASI-75 will be determined where PASI-50 is defined as a 50% or greater fall in PASI score from baseline and PASI-75 is defined as a 75% or greater fall in PASI score.

### EXAMPLE 1

### Trk Inhibition Test

Candidate compounds for the inhibition of keratinocyte proliferation may be selected according to their ability to inhibit the tyrosine kinase activity associated with trkA. Upon binding of NGF, trkA undergoes autophosphorylation as a result of the activation of its tyrosine kinase domain (Kaplan et al. Nature 350:158-160, 1991).

The candidate compounds are tested for their ability to inhibit the kinase activity of baculovirus-expressed human trkA cytoplasmic domain using an ELISA-based assay as previously described (Angeles et al., Anal. Biochem. 236: 49-55, 1996). Briefly, the 96-well microtiter plate is coated with substrate solution (recombinant human phospholipase C-γ1/glutathione S-transferase fusion protein (Rotin et al., EMBO J., 11: 559-567, 1992). Inhibition studies are performed in 100 μl assay mixtures containing 50 mM Hepes, pH 7.4, 40 μM ATP, 10 mM MnCl₂, 0.1% BSA, 2% DMSO, and various concentrations of inhibitor. The reaction is initiated by addition of trkA kinase and allowed to proceed for 15 minutes at 37° C. An antibody to phosphotyrosine (UBI) is then added, followed by a secondary enzyme-conjugated antibody, alkaline phosphatase-labelled goat anti-mouse IgG (Bio-Rad). The activity of the bound enzyme is measured via an amplified detection system (Gibco-BRL). Inhibition data is analyzed using the signioidal dose-response (variable slope) equation in GraphPad Prism. The concentration that results in 50% inhibition of kinase activity is referred to as “IC₅₀”.

### EXAMPLE 2

A 70 year old white male patient with a history of renal and prostate cancer, arthritic pain and chronic bronchitis presented with severe psoriasis in the ankle region. The patient started treatment with lestaurtinib at a dosage of 60 mg bid. Lestaurtinib was administered twice daily as an oral solution at a concentration of 25 mg/mL in polysorbate 80 NF (10 mL) and propylene glycol USP (10 mL), diluted in juice. The following juices are approved for use to administer lestaurtinib: grape, pineapple, apple, V8® 100% vegetable juice, and orange juice (pulp free). After 29 days from initiation, the dosage was increased to 80 mg bid. On day 60 from the beginning of treatment, the patient was observed to have relative remission of psoriasis.

### EXAMPLE 3

A patient is selected having moderate to severe psoriasis characterized by body surface area involvement of 10% or greater. The patient is administered treatment of a pharmaceutical composition of LESTAURTINIB at a dosage of 60 mg bid.

The patient undergoing treatment is analyzed by a dermatologist or an experienced physician for each area of the body that is afflicted with psoriasis. Each area of the body is rated for erythema, scaling, and thickness for the average plaque or the overall condition of plaques in that region. The plaques are rated as they are actually seen on the day of examination and not in comparison with baseline condition.

A PASI score is determined for the patient at study baseline and at final study assessment. It will be determined whether the patient falls into the class of PASI-50, defined as a 50% or greater fall in PASI score from baseline, or PASI-75, defined as a 75% or greater fall in PASI score.

As those skilled in the art will appreciate, numerous modifications and variations of the present invention are possible in light of the above teachings. It is therefore understood that within the scope of the appended claims, the invention may be practiced otherwise than as specifically described herein, and the scope of the invention is intended to encompass all such variations.

What is claimed:

1. A method of treating a proliferative skin disease, comprising administering to a patient a therapeutically effective amount of a compound that is a trk inhibitor.

2. The method of claim 1, wherein the trk inhibitor is a compound having the formula: 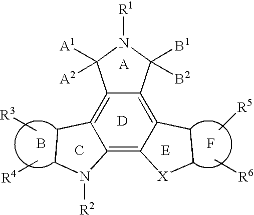
or a stereoisomer or pharmaceutically acceptable salt form thereof, wherein:
rings B and F, independently, are phenyl or heteroaryl;
R is H; alkyl; aryl; arylalkyl; heteroaryl; heteroarylalkyl; —COR⁹; —OR¹⁰; —CONR⁷R⁸; —NR⁷R⁸; —(CH₂)ₚNR⁷R⁸; —(CH₂)ₚOR¹⁰; —O(CH₂)ₚOR¹⁰; or —O(CH₂)ₚNR⁷R⁸;
R² is H; —SO₂R⁹; —CO₂R⁹; —COR⁹; alkyl having 1 to 8 carbons; alkenyl having 2 to 8 carbons; alkynyl having 2 to 8 carbons; or a monosaccharide having 5 to 7 carbons,
wherein each hydroxyl group of the monosaccharide, independently, is optionally replaced by an alkyl having 1 to 4 carbons, alkylcarbonyloxy having 2 to 5 carbons or alkoxy having 1 to 4 carbons; and
wherein the alkyl, alkenyl, or alkynyl groups are optionally substituted with one to three R²⁷ groups;
R³, R⁴, R⁵ and R⁶, independently, are H; aryl; heteroaryl; F; Cl; Br; I; —CN; CF₃; —NO₂; —OR¹⁰; —O(CH₂)ₚNR⁷R⁸; —OCOR⁹; —OCONHR⁹; —CH₂OR¹⁴; —NR⁷R⁸; —NR¹⁰COR⁹; —NR¹⁰CONR⁷R⁸; —S(O)_{y}R¹¹; —CO₂R⁹; —COR⁹; —CONR⁷R⁸; —CHO; —CH═NOR¹¹; —CH═NR⁹; —CH═NNR¹²R¹³; —(CH₂)ₚS(O)_{y}R⁹; —CH₂SR¹⁵; —CH₂S(O)_{y}R¹⁴; —(CH₂)ₚNR⁷R⁸; —(CH₂)ₚNHR¹⁴; alkyl having 1 to 8 carbons; alkenyl having 2 to 8 carbons; or alkynyl having 2 to 8 carbons;
wherein the alkyl, alkenyl, or alkynyl groups are optionally substituted with one to three R²⁷ groups;
X is:
alkylene having 1 to 3 carbons optionally substituted with at least one of OH; ═O; ═NOR¹¹; OR¹¹; —OCOR⁹; —OCONR⁷R⁸; —O(CH₂)ₚNR⁷R⁸; —O(CH₂)ₚOR¹⁰; aryl; arylalkyl; heteroaryl; —SO₂R⁹; —CO₂R⁹; —COR⁹; alkyl having 1 to 8 carbons; alkenyl having 2 to 8 carbons; alkynyl having 2 to 8 carbons; or a monosaccharide having 5 to 7 carbons,
wherein each hydroxyl group of the monosaccharide, independently, is optionally replaced by an alkyl having 1 to 4 carbons, alkylcarbonyloxy having 2 to 5 carbons or alkoxy having 1 to 4 carbons; and
wherein the alkyl, alkenyl, or alkynyl groups are optionally substituted with one to three R²⁷ groups;
—O—; —S(O)_{y}—; N(R¹⁶); —CH₂Z—; —Z—CH₂—; or —CH₂ZCH₂—;
wherein Z is C(OR¹¹)(R¹¹), O, S, C(═O), C(═NOR¹¹), or NR¹¹; or CHR¹⁶;
wherein R¹⁶ and R² can optionally be combined together to form a linking furan via its 2 and 5 positions and wherein positions 2 and 5 of the linking furan are optionally substituted with R²⁸ and R²⁹, respectively; and position 3 of the linking furan is disubstituted with R¹⁷ and R¹⁸;
A¹ and A², independently, are H, —OR¹¹, —SR¹¹, or —N(R¹¹)₂; or, combined together, form a moiety that is ═O, ═S, or ═NR¹¹; and
B¹ and B² independently, are H, —OR¹¹, —SR¹¹, or —N(R¹¹)₂; or, combined together, form a moiety that is ═O, ═S, or ═NR¹¹;
with the proviso that at least one of the pair of A¹ and A², or B¹ and B² is combined together to form ═O;
R⁷ and R⁸, independently, are H or alkyl of 1 to 4 carbons, or, together with the nitrogen to which they are attached, form a 5 to 7 membered heterocycloalkyl;
R⁹ is alkyl having 1 to 4 carbons, aryl, or heteroaryl;
R¹⁰ is H or alkyl having 1 to 4 carbons;
R¹¹ is H, alkyl having 1 to 4 carbons, aryl having 6 to 10 carbons, or heteroaryl;
R¹² and R¹³, independently, are H, alkyl, aryl having 6 to 10 carbons, or heteroaryl; or, together with the nitrogen to which they are attached, form a 5 to 7 membered heterocycloalkyl;
R¹⁴ is the residue of an amino acid after the hydroxyl group of the carboxyl group is removed;
R¹⁵ is alkyl having 1 to 4 carbons;
R¹⁶ is lower alkyl, aryl, or heteroaryl;
R¹⁷ is OH, O-n-alkyl having 1 to 6 carbons, or O-acyl having 2 to 6 carbons;
R¹⁸ is H; alkyl having 1 to 4 carbons; CONHC₆H₅; CH₂Y,
wherein Y is OR¹⁹, SOR²⁰, NR²¹R²², or SR²³; N₃; CO₂R¹⁵; S-Glc; CONR²⁴R²⁵; CH═NNHCONH₂; CONHOR¹⁰; CH═NOR¹⁰; CH═NNHC(═NH)NH₂; 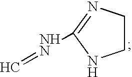
CH═NN(R²⁶)₂; or CH₂NHCONHR¹⁶;
or R¹⁷ and R¹⁸ can optionally be combined together to form —CH₂NHCO₂—, —CH₂OC(CH₃)₂O—, ═O, or —CH₂N(CH₃)CO₂—; and
R¹⁹ is H, alkyl having 1 to 4 carbons, or acyl having 2 to 5 carbons;
R²⁰ is alkyl having 1 to 4 carbons, aryl, or a heterocycloalkyl group including a nitrogen atom;
R²¹ and R²², independently, are H, alkyl having 1 to 4 carbons, Pro, Ser, Gly, Lys, or acyl having 2 to 5 carbons, with the proviso that only one of R²¹ and R²² is Pro, Ser, Gly, Lys or acyl;
R²³ is an aryl, alkyl having 1 to 4 carbons, or a heterocycloalkyl group that includes a nitrogen atom;
R²⁴ and R²⁵, independently, are H; alkyl having 1 to 6 carbons; phenyl; or hydroxyalkyl of 1-6 carbons; or, together with the nitrogen to which they are attached, form a 5 to 7 membered heterocycloalkyl;
R²⁶ is aryl;
R²⁷ is aryl; heteroaryl; F; Cl; Br; I; —CN; —NO₂; —OR¹⁰; —O(CH₂)ₚNR⁷R⁸; —OCOR⁹; —OCONHR⁹; O-tetrahydropyranyl; —NR⁷R⁸; —NR¹⁰COR⁹; —NR¹⁰CO₂R⁹; —NR¹⁰CONR⁷R⁸; —NHC(═NH)NH₂; —NR¹⁰SO₂R⁹; —S(O)_{y}R¹¹; —CO₂R⁹; —CONR⁷R⁹; —CHO; —COR⁹; —CH₂OR⁷; —CH═NNR¹²R¹³; —CH═NOR¹¹; —CH═NR⁹; —CH═NNHCH(N═NH)NH₂; —SO₂NR¹²R¹³; —PO(OR¹¹)₂; or —OR¹⁴;
R²⁸ is alkyl having from 1 to 4 carbons, alkoxy having from 1 to 4 carbons, arylalkyl having from 6 to 10 carbons, —(CH₂)ₚOR¹⁰, —(CH₂)ₚOC(═O)NR⁷R⁸, or —(CH₂)ₚNR⁷R⁸;
R²⁹ is H, alkyl having from 1 to 4 carbons, alkoxy having from 1 to 4 carbons, arylalkyl having from 6 to 10 carbons, —(CH₂)ₚOR¹⁰, —(CH₂)ₚOC(═O)NR⁷R⁸, or —(CH₂)ₚNR⁷R⁸;
p is an integer from 1 to 4; and
y is 0, 1 or 2.

3. The method of claim 1, wherein the trk inhibitor is a compound having the formula: 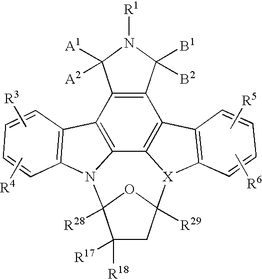
or a stereoisomer or pharmaceutically acceptable salt form thereof, wherein:
R¹ is H; alkyl; phenyl; arylalkyl having 7 to 10 carbons; 5-6 membered heteroaryl; heteroarylalkyl; —COR⁹; —OR¹⁰; —CONR⁷R⁸; —NR⁷R⁸; —(CH₂)ₚNR⁷R⁸; —(CH₂)ₚOR¹⁰; —O(CH₂)ₚOR¹⁰; or —O(CH₂)ₚNR⁷R⁸;
R³, R⁴, R⁵ and R⁶, independently, are H; phenyl; 5-6 membered heteroaryl; F; Cl; Br; I; —CN; CF₃; —NO₂; —OR¹⁰; —O(CH₂)ₚNR⁷R⁸; —OCOR⁹; —OCONHR⁹; —CH₂OR¹⁴; —NR⁷R⁸; —NR¹⁰COR⁹; —NR¹⁰CONR⁷R⁸; —S(O)_{y}R¹¹; —CO₂R⁹; —COR⁹; —CONR⁷R⁸; —CHO; —CH═NOR¹¹; —CH═NR¹¹; —CH═NNR¹²R¹³; —(CH₂)ₚS(O)R_{y}R⁹; —CH₂SR¹⁵; —CH₂S(O)_{y}R¹⁴; —(CH₂)ₚNR⁷R⁸; —(CH₂)ₚNHR¹⁴; alkyl having 1 to 8 carbons; alkenyl having 2 to 8 carbons; or alkynyl having 2 to 8 carbons;
wherein the alkyl, alkenyl, or alkynyl groups are optionally substituted with one to three R²⁷ groups;
X is —CH— or N;
A¹ and A², independently, are H, —OR¹¹, —SR¹¹, or —N(R¹¹)₂; or, combined together, form a moiety that is ═O, ═S, or ═NR¹¹; and
B¹ and B² independently, are H, —OR , —SR¹¹, or —N(R¹¹)₂; or, combined together, form a moiety that is ═O, ═S, or ═NR¹¹;
with the proviso that at least one of the pair of A¹ and A², or B¹ and B² is combined together to form ═O;
R⁷ and R⁸, independently, are H or alkyl of 1 to 4 carbons, or, together with the nitrogen to which they are attached, form a 5 to 7 membered heterocycloalkyl;
R⁹ is alkyl having 1 to 4 carbons, aryl, or heteroaryl;
R¹⁰ is H or alkyl having 1 to 4 carbons;
R¹¹ is H, alkyl having 1 to 4 carbons, aryl having 6 to 10 carbons, or heteroaryl;
R¹² and R¹³, independently, are H, alkyl, aryl having 6 to 10 carbons, or heteroaryl;
or, together with the nitrogen to which they are attached, form a 5 to 7 membered heterocycloalkyl;
R¹⁴ is the residue of an amino acid after the hydroxyl group of the carboxyl group is removed;
R¹⁵ is alkyl having 1 to 4 carbons;
R¹⁶ is lower alkyl, aryl, or heteroaryl;
R¹⁷ is OH, O-n-alkyl having 1 to 6 carbons, or O-acyl having 2 to 6 carbons;
R¹⁸ is H; alkyl having 1 to 4 carbons; CONHC₆H₅; CH₂Y,
wherein Y is OR¹⁹, SOR²⁰, NR²¹R²², or SR²³; N₃; CO₂R¹⁵; S-Glc; CONR²⁴R²⁵; CH═NNHCONH₂; CONHOR¹⁰; CH═NOR¹⁰; CH═NNHC(═NH)NH₂; 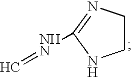
CH═NN(R²⁶)₂; or CH₂NHCONHR¹⁶;
or R¹⁷ and R¹⁸ are optionally combined together to form —CH₂NHCO₂—, —CH₂OC(CH₃)₂O—, ═O, or —CH₂N(CH₃)CO₂—; and
R¹⁹ is H, alkyl having 1 to 4 carbons, or acyl having 2 to 5 carbons;
R²⁰ is alkyl having 1 to 4 carbons, aryl, or a heterocycloalkyl group including a nitrogen atom;
R²¹ and R²², independently, are H, alkyl having 1 to 4 carbons, Pro, Ser, Gly, Lys, or acyl having 2 to 5 carbons, with the proviso that only one of R²¹ and R²² is Pro, Ser, Gly, Lys or acyl;
R²³ is an aryl, alkyl having 1 to 4 carbons, or a heterocycloalkyl group that includes a nitrogen atom;
R²⁴ and R²⁵, independently, are H; alkyl having 1 to 6 carbons; phenyl; or hydroxyalkyl of 1-6 carbons; or, together with the nitrogen to which they are attached, form a 5 to 7 membered heterocycloalkyl;
R²⁶ is aryl;
R²⁷ is aryl; heteroaryl; F; Cl; Br; I; —CN; —NO₂; —OR¹⁰; —O(CH₂)ₚNR⁷R⁸; —OCOR⁹; —OCONHR⁹; O-tetrahydropyranyl; —NR⁷R⁸; —NR¹⁰COR⁹; —NR¹⁰CO₂R⁹; —NR¹⁰CONR⁷R⁸; —NHC(═NH)NH₂; —NR¹⁰SO₂R⁹; —S(O)_{y}R¹¹; —CO₂R⁹; —CONR⁷R⁸; —CHO; —COR⁹; —CH₂OR⁷; —CH═NNR¹²R¹³; —CH═NOR¹¹; —CH═NR⁹; —CH═NNHCH(N═NH)NH₂; —SO₂NR¹²R¹³; —PO(OR¹¹)₂; or —OR¹⁴;
R²⁸ is alkyl having from 1 to 4 carbons, alkoxy having from 1 to 4 carbons, arylalkyl having from 6 to 10 carbons, —(CH₂)ₚOR¹⁰, —(CH₂)ₚOC(═O)NR⁷R⁸, or —(CH₂)ₚNR⁷R⁸;
R²⁹ is H, alkyl having from 1 to 4 carbons, alkoxy having from 1 to 4 carbons, arylalkyl having from 6 to 10 carbons, —(CH₂)ₚOR¹⁰, —(CH₂)ₚOC(═O)NR⁷R⁸, or —(CH₂)ₚNR⁷R⁸;
p is an integer from 1 to 4; and
y is 0, 1 or 2.

4. The method of claim 3, wherein the trk inhibitor is a compound having the formula:

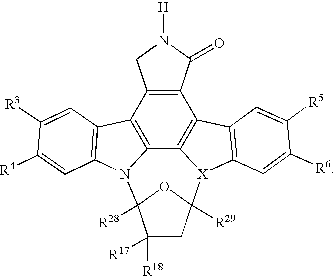

5. The method of claim 1, wherein the trk inhibitor is a compound having the formula: 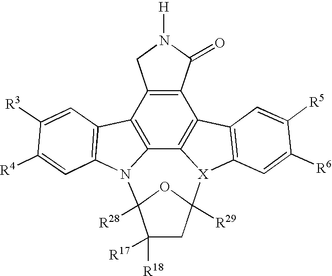
or a stereoisomer or pharmaceutically acceptable salt form thereof, wherein:
R³, R⁴, R⁵ and R⁶, independently, are H; phenyl; F; Cl; —OR¹⁰; —NR⁷R⁸; —CHO; —(CH₂)ₚNR⁷R⁸; or alkyl having 1 to 8 carbons;
wherein the alkyl group is optionally substituted with one to three R²⁷ groups;
X is —CH— or N;
R⁷ and R⁸, independently, are H or alkyl of 1 to 4 carbons, or, together with the nitrogen to which they are attached, form a 5 to 7 membered heterocycloalkyl;
R⁹ is alkyl having 1 to 4 carbons, aryl, or heteroaryl;
R¹⁰ is H or alkyl having 1 to 4 carbons;
R¹¹ is H, alkyl having 1 to 4 carbons, aryl having 6 to 10 carbons, or heteroaryl;
R¹² and R¹³, independently, are H, alkyl, aryl having 6 to 10 carbons, or heteroaryl; or, together with the nitrogen to which they are attached, form a 5 to 7 membered heterocycloalkyl;
R¹⁴ is the residue of an amino acid after the hydroxyl group of the carboxyl group is removed;
R¹⁷ is OH, O-n-alkyl having 1 to 6 carbons, or O-acyl having 2 to 6 carbons;
R¹⁸ is H, alkyl having 1 to 4 carbons, CONHC₆H₅; CH₂OH; CH₂OCH₃; CH₂OC(CH₃)₃; CH₂NH₂; CO₂CH₃; CONR²⁴R²⁵;
R²⁴ and R²⁵, independently, are H; alkyl having 1 to 6 carbons; phenyl; or hydroxyalkyl of 1-6 carbons; or, together with the nitrogen to which they are attached, form a 5 to 7 membered heterocycloalkyl;
R²⁷ is aryl; heteroaryl; F; Cl; Br; I; —CN; —NO₂; —OR¹⁰; —O(CH₂)ₚNR⁷R⁸; —OCOR⁹; —OCONHR⁹; O-tetrahydropyranyl; —NR⁷R⁸; —NR¹⁰COR⁹; —NR¹⁰CO₂R⁹; —NR¹⁰CONR⁷R⁸; —NHC(═NH)NH₂; —NR¹⁰SO₂R⁹; —S(O)_{y}R¹¹; —CO₂R⁹; —CONR⁷R⁸; —CHO; —COR⁹; —CH₂OR⁷; —CH═NNR¹²R¹³, —CH═NOR¹¹; —CH═NR⁹; —CH═NNHCH(N═NH)NH₂; —SO₂NR¹²R¹³; —PO(OR¹¹)₂; or —OR¹⁴;
R²⁸ is alkyl having from 1 to 4 carbons, alkoxy having from 1 to 4 carbons, arylalkyl having from 6 to 10 carbons, —(CH₂)ₚOR¹⁰, —(CH₂)ₚOC(═O)NR⁷R⁸, or —(CH₂)ₚNR⁷R⁸;
R²⁹ is H, alkyl having from 1 to 4 carbons, alkoxy having from 1 to 4 carbons, arylalkyl having from 6 to 10 carbons, —(CH₂)ₚOR¹⁰, —(CH₂)ₚOC(═O)NR⁷R⁸, or —(CH₂)ₚNR⁷R⁸;
p is an integer from 1 to 4; and
y is 0, 1 or 2.

6. The method of claim 1, wherein the trk inhibitor is a compound having the formula: 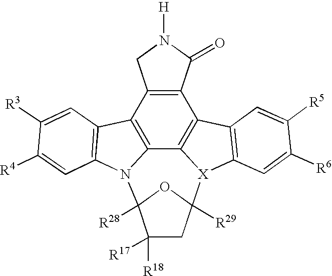
or a stereoisomer or pharmaceutically acceptable salt form thereof, wherein:
X is CH or N;
R³, R⁴, R⁵, and R⁶, independently, are H, Cl, alkyl of 1-4 carbons, —OR¹⁰, CH₂OH, CHO, NH₂, CH₂NH₂, CH₂OCH, CH₂OC(CH₃)₃, or CONH₂;
R¹⁰ is H or alkyl having 1 to 4 carbons;
R¹⁷ is OH, O-n-alkyl having 1 to 4 carbons;
R¹⁸ is H, CH₂OH, CO₂CH₃, CO₂CH₃, CO₂CH₂CH₃, CO₂CH₂CH₂CH₃, or CO₂CH(CH₃)₂; or
R²⁸ is CH₃; and
R²⁹ is H or CH₃.

7. The method of claim 1, wherein the trk inhibitor is a compound having the formula:

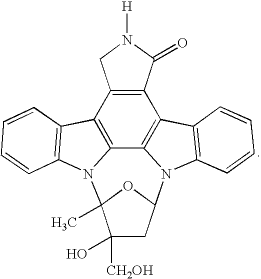

8. The method of claim 1, wherein the trk inhibitor is a compound having the formula:

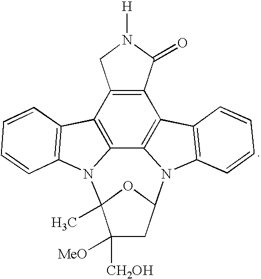

9. The method of claim 1, wherein the proliferative skin disease is actinic keratosis, basal cell carcinoma, squamous cell carcinoma, fibrous histiocytoma, dermatofibrosarcoma protuberans, hemangioma, nevus flammeus, xanthoma, Kaposi's sarcoma, mastocytosis, mycosis fungoides, lentigo, nevocellular nevus, lentigo maligna, malignant melanoma, metastatic carcinoma or psoriasis.

10. The method of claim 2, wherein the proliferative skin disease is actinic keratosis, basal cell carcinoma, squamous cell carcinoma, fibrous histiocytoma, dermatofibrosarcoma protuberans, hemangioma, nevus flammeus, xanthoma, Kaposi's sarcoma, mastocytosis, mycosis fungoides, lentigo, nevocellular nevus, lentigo maligna, malignant melanoma, metastatic carcinoma or psoriasis.

11. The method of claim 7, wherein the proliferative skin disease is actinic keratosis, basal cell carcinoma, squamous cell carcinoma, fibrous histiocytoma, dermatofibrosarcoma protuberans, hemangioma, nevus flammeus, xanthoma, Kaposi's sarcoma, mastocytosis, mycosis fungoides, lentigo, nevocellular nevus, lentigo maligna, malignant melanoma, metastatic carcinoma or psoriasis.

12. The method of claim 8, wherein the proliferative skin disease is actinic keratosis, basal cell carcinoma, squamous cell carcinoma, fibrous histiocytoma, dermatofibrosarcoma protuberans, hemangioma, nevus flammeus, xanthoma, Kaposi's sarcoma, mastocytosis, mycosis fungoides, lentigo, nevocellular nevus, lentigo maligna, malignant melanoma, metastatic carcinoma or psoriasis.

13. The method of claim 1, wherein the proliferative skin disease is psoriasis.

14. The method of claim 2, wherein the proliferative skin disease is psoriasis.

15. The method of claim 7, wherein the proliferative skin disease is psoriasis.

16. The method of claim 8, wherein the proliferative skin disease is psoriasis.
